(19) United States
(12) Patent Application Publication
(10) Pub. No.: US 2009/0183281 A1
(43) Pub. Date: Jul. 16, 2009

(54) Use of histone chaperone activity of agrobacterium 6b protein

(75) Inventors: Yasunori Machida, Nagoya-shi (JP); Kenzo Nakamura, Nagoya-shi (JP)

Correspondence Address: OLIFF & BERRIDGE, PLC, P.O. BOX 320850, ALEXANDRIA, VA, 22320-4850, US

(73) Assignee: NATIONAL UNIVERSITY CORPORATION NAGOYA UNIVERSITY, NAGOYA-SHI (JP)

(21) Appl. No.: 12/227,591

(22) PCT Filed: May 25, 2006
(86) PCT No.: PCT/JP2006/311030

§ 371 Date: Dec. 11, 2008

(51) Int. Cl. C12N 15/82; A01H 5/00; C07K 14/415

(52) U.S. Cl. 800/290; 800/298; 530/370; 536/25.3

(57) ABSTRACT

The present invention provides a technology for modifying the gene expression of a plant body in combination with using histone-chaperon-activity of protein 6b. According to the invention, gene expression is changed or modified globally.

### TECHNICAL FIELD

The present invention relates to use of histone-chaperone-activity of Agrobacterium 6b protein.

### BACKGROUND ART

Agrobacterium tumefaciens and A. vitis strains that harbor the Ti plasmids induce crown gall tumors upon infection of dicotyledonous plants. T-DNAs from most Ti plasmids contain the three well-characterized genes ipt (tmr), iaaM (tms1) and iaah (tms2), which are involved in biosynthesis of cytokinin and auxin, respectively, and responsible for the formation of the crown gall tumors. This region also encodes a gene called 6b, which exhibits an oncogenic effect on certain plant species (Hooykaas et al. 1988; Tinland et al. 1989). The 6b genes from various Ti plasmids stimulate ipt- and iaaM/iaaH-induced division of cells (Tinland et al. 1989; Wabiko and Minemura 1996) and induce the formation of shooty calli when discs from leaves that express 6b gene from pTiAKE10 (AK-6b) are incubated in the absence of exogenous phytohormones in culture medium (Wabiko and Minemura 1996). Therefore, the AK-6b gene appears to play a role in the proliferation of plant cells, which might be related to the action of the plant growth regulators auxin and cytokinin (Kitakura et al. 2002). It has been reported that transgenic tobacco plants that express 6b genes from various sources show abnormal leaf morphology. Transgenic plants of Nicotiana rustica in which the T-6b gene (from pTiTm4) is driven by the heat-shock promoter generate tubular leaves upon heat shock treatment (Tinland et al. 1992), and the transgenic tobacco plants that express AK-6b develop small leaf-like structures from veins of the abaxial leaf surface, some of which are extremely asymmetric along the midvein (Wabiko and Minemura 1996).

Recently, Helfer et al. (2003) reported that AB-6b (from pTiAB4) transgenic tobacco plants formed extra cell layers in the abaxial side of leaves and displayed alterations in flower morphology, and that AB-6b transgenic Arabidopsis plants generated radial symmetrical tubes on the abaxial side of the leaves. Northern blot analysis of cell cycle genes in AB-6b-transformed leaves, however, showed no significant difference in levels of transcripts of these genes compared with those in untransformed leaves (Helfer et al. 2003). However, the relationship between severity of phenotypes generated by the 6b gene, and levels of transcripts of genes related to cell division and organ development, has yet to be extensively investigated. It also remains to be examined how the phenotypes are directly related to cell division. In addition, expression of AK-6b affects levels of accumulation of various metabolites including phenolics in plants (Gális et al. 2004: Kakiuchi et al. 2005), though the genetic basis for such effects is unclear. The 6b protein has been shown to be localized to plant nuclei and associated with a nuclear protein of tobacco named NtSIP1 (Kitakura et al. 2002). NtSIP1 has an amino acid sequence that is similar to a tri-helix motif, which is known to be a DNA-binding sequence in the rice transcription factor GT-2 (Dehesh et al. 1992); and promotes nuclear localization of the 6b protein. A chimeric 6b protein that is fused to the DNA-binding domain of yeast GAL4 protein activates transcription of a reporter gene in tobacco cells. However, it has not been examined whether nuclear localization of 6b protein is essential for the generation of 6b-related phenotypes. Recently, it has been reported the T-6b protein moves through leaf cells (Grémillon et al. 2004).

Further, protein 6b, encoded by the T-DNA from the pathogen Agrobacterium, which can induce crown gall tumours on plants, stimulates the plant-hormone-independent division of cells in vitro and aberrant cell growth in 6b-expressing transgenic plants 1-7. Evolutionary conservation of the 6b gene in the T-DNA of all Ti plasmids suggests a crucial role for 6b in the induction of cell division and in the formation of plant tumours. We showed previously that nuclear localization of 6b is essential for the 6b-induced phenotype and that 6b binds plant nuclear proteins 8. To date, however, the molecular functions of 6b have remained unknown.

Cells of Agrobacterium tumefaciens that harbour a Ti plasmid induce the formation of tumours, known as crown galls, on many dicotyledonous plants. Upon infection by the bacterium, a specific region of the Ti plasmid (the T-DNA) is transferred to the plant cell and is integrated into the chromosomal DNA in the nucleus. Cells that have chromosomally integrated T-DNA can divide autonomously to generate a tumour, which appears to be a consequence, for the most part, of the expression of genes that are responsible for the biosynthesis of auxin (aux1/iaaM/tms1 and aux2/iaaH/tms2) and cytokinin (cyt/ipt/tmr) 10. In addition to these genes, a gene designated 6b, which is localized at the tm1 locus1,2 and has been found in the T-DNA of all examined strains of A. tumefaciens and A. vitis11, appears to play a role in the proliferation of plant cells6,8, although the effects of 6b are relatively weak. The predicted amino acid sequence of 6b is somewhat homologous to those of a number of proteins encoded by other genes, such as rolB and ORF13 in Ri plasmids, which also cause aberrant growth and abnormal root and shoot morphology12,13. Various hypotheses have been proposed to explain the effects of these genes on the growth of plant cells3-6,14,15. There are some discrepancies among previously reported results but it is generally accepted that 6b affects cell proliferation and shoot development by modulating the actions of cytokinin and/or auxin. The molecular mechanisms whereby 6b acts remain, however, to be characterized.

### DISCLOSURE OF THE INVENTION

An object of the present invention is to provide a technology for modifying the gene expression of plant body in combination with using histone-chaperon-activity of protein 6b. Another object of the present invention is to provide a technology for globally modifying the gene expression of plant body in combination with using histone-chaperon-activity of protein 6b. Another object of the present invention is to provide a technology for modifying the chromatin structure of plant body.

In the present teaching, we focused on the leaf abnormality generated by expression of AK-6b because such an abnormality is consistently observed both in transgenic tobacco and Arabidopsis that express the AK-6b gene: in particular upwardly curled leaves were commonly found at early developmental stages of leaves of both plant species. Anatomical studies and in situ hybridization of M-phase-specific genes with leaves of transgenic tobacco suggested that the division competence of cells is enhanced in the abaxial side of the transgenic leaves. Using a glucocorticoid receptor-fused AK-6b, we showed that nuclear localization is essential for the phenotypes generated by AK-6b including upward curling of leaves and hormone-free callus formation.

Further, in the present teaching we disclose here that 6b binds to histone H3 and acts as a histone chaperone in vitro. Mutant 6b proteins, deficient in histone-chaperone activity, lack division-stimulating activity. Our results suggest a strong relationship between alterations in chromatin structure and the expression of growth regulating genes on the one hand and induction of aberrant cell proliferation on the other. Thus, the activity of 6b as a histone chaperone might be important for the long-term maintenance of crown galls caused by genetic transformation. Moreover, we show here that 6b has histone-chaperon-activity and our observation suggests the importance of alterations in the structures of plant chromatin that are induced by 6b in unregulated growth of cells in crown galls.

Based on the foregoing findings, the present inventors have been accomplished the present invention. In a more specific aspect, the present invention provides the following:

(1) A modification method of modifying gene expression of a plant body, said modification method comprising at least one of the steps of:
(a) modifying the plant body to allow production of Agrobacterium 6b protein or a modified body thereof; and
(b) modifying the plant body to allow change in expression level of Agrobacterium 6b protein in an enhancing or repressing direction.
(2) A modification method in accordance with claim 1, said method modifying expression of at least one gene regulated directly or indirectly by Agrobacterium 6b protein.
(3) A modification method in accordance with (2), wherein the at least one gene regulated directly or indirectly by Agrobacterium 6b protein is selected from a group of genes in an IAA gene family and a TCP gene family.
(4) A modification method in accordance with (3), wherein the genes in the IAA gene family include IAA1, IAA2, IAA3/SHY2, IAA4, IAA6, IAA8, IAA14/SLR1, IAA16, IAA17AXR3, IAA27/PAP2, IAA30 and IAA34.
(5) A modification method in accordance with (4), wherein the genes in the TCP gene family include TCP4, TCP5, TCP10 and TCP20.
(6) A modification method in accordance with any one of (1) through (5), wherein the modification of the gene expression includes a change in phenotype of the plant body.
(7) A modification method in accordance with any one of (1) through (6), wherein the modification of the gene expression includes enhanced or repressed gene expression of a gene out of the IAA gene family and the TCP gene family.
(8) A modification method in accordance with any one of (1) through (7), wherein the modification of the gene expression includes at least one of promotion of proliferation, inhibition of proliferation, promotion of growth, inhibition of growth, and improved yield of the plant body.
(9) A modification method in accordance with any one of (1) through (8), wherein Agrobacterium 6b protein is identical with or substantially equivalent to a protein having an amino acid showed in SEQ ID NO: 2.
(10) A modification method in accordance with any one of (1) through (9), wherein the modification of Agrobacterium 6b protein includes modification of at least either of an amino acid sequence in an acidic amino acid region at a C-terminal and an amino acid sequence in an adjoining amino acid region adjacent to the acidic amino acid region at the C-terminal.
(11) A modification method in accordance with any one of (1) through (10), wherein the modification of Agrobacterium 6b protein includes modification of an amino acid sequence in an amino acid region other than the acidic amino acid region and the adjoining amino acid region at the C-terminal.
(12) A modification method in accordance with any one of (1) through (11), said method further comprising the steps of:
(c) modifying the plant body to allow production of a histone protein or a modified body thereof, which interacts with Agrobacterium 6b protein; and
(d) modifying the plant body to allow change in expression level of the histone protein in an enhancing or repressing direction.
(13) A modification method in accordance with 1(12), wherein the histone protein is histone H3 protein.
(14) A modification method in accordance with either one of (12) and (13), wherein the modification of the histone protein includes modification of an amino acid sequence in at least either of a folding region and a non-folding region of histone H3 protein.
(15) A modification method in accordance with any one of (1) through (14), wherein the plant body is selected from the group consisting of a plant cell, a cultured plant cell, a protoplast, a callus, a plant tissue, a plant organ, an individual plant body, and a reproduced plant body.
(16) A modification method in accordance with any one of (1) through (15), wherein the plant body is a dicotyledoneae.
(17) A production method of producing a plant body with gene expression modified by a modification method in accordance with any one of (1) through (16).
(18) A production method in accordance with (17), said production method comprising the step of:
proliferating, growing or breeding the plant body.
(19) A plant body having modified gene expression, said plant body being modified to allow at least either of expression of Agrobacterium 6b protein or its modified body and change of an expression level of Agrobacterium 6b protein in an enhancing or repressing direction.
(20). A plant body in accordance with (19), said plant body being further modified to allow at least either of expression of a histone protein or a modified body thereof, which interacts with Agrobacterium 6b protein, and change in expression level of the histone protein in an enhancing or repressing direction.
(21) A plant body in accordance with either one of (19) and (20), said plant body having modified expression of at least one gene selected from genes in an IAA gene family and a TCP gene family.
(22) A gene expression method of expressing a gene of a plant body, said gene expression method comprising the steps of: (a) preparing a plant body modified to have a first modification and a second modification, the first modification being attained either by enhancement or repression of an expression level of a selected internal gene or by introduction of an exogenous gene coding a selected protein, the second modification being attained by at least either of expression of Agrobacterium 6b protein or its modified body and change of an expression level of Agrobacterium 6b protein in an enhancing or repressing direction; and (b) proliferating, growing, or breeding the plant body.
(23) A gene expression method in accordance with (22), wherein said step (a) substitutes at least one gene having expression regulated directly or indirectly by Agrobacterium 6b protein with the exogenous gene coding the selected protein.
(24) A gene expression method in accordance with either one of (22) and (23), wherein said step (a) suppresses expression of at least one gene regulated directly or indirectly by Agrobacterium 6b protein.
(25) A gene expression method in accordance with either one of (23) and (24), wherein the at least one gene having expression regulated directly or indirectly by Agrobacterium 6b protein is selected from genes in an IAA gene family and a TCP gene family.
(26) A gene expression method in accordance with any one of (22) through (25), wherein said step (a) introduces at least two different genes into to a host chromosome.
(27) A gene expression method in accordance with any one of (22) through (26), wherein the plant body expresses a modified body of histone H3 protein.
(28) A gene expression method in accordance with any one of (22) through (27), wherein said step (a) genetically modifies a plant body producing Agrobacterium 6b protein or a modified body thereof.
(29) A gene expression method in accordance with any one of (22) through (27), wherein said step (a) genetically modifies the plant body and introduces a gene of Agrobacterium 6b protein into the genetically modified plant body to allow production of Agrobacterium 6b protein.
(30). A substance production method of producing a substance by adopting either of a modification method in accordance with any one of (1) through (16) and a gene expression method in accordance with any one of claims (22) through (29).
(31) A substance production method in accordance with (29), wherein the substance is selected from the group consisting of proteins, low-molecular organic compounds, sugars, and lipids.
(32) A plant chromatin structure having Agrobacterium 6b protein or a modified body thereof.
(33) A plant chromatin structure in accordance with (32), said plant chromatin structure further having a histone protein or a modified body thereof, which interacts with Agrobacterium 6b protein.
(34) A plant body comprising said plant chromatin structure in accordance with either one of (32) and (33).
(35) A production method of producing a modified plant chromatin structure, said production method comprising the step of: bringing Agrobacterium 6b protein or a modified body thereof in contact with a chromatin structure-producing material in a chromatin structure-producing environment.
(36) A production method in accordance with (35), wherein said step bringing Agrobacterium 6b protein or the modified body thereof in contact with the chromatin structure-producing material in the presence of histone H3 protein in the chromatin structure-producing environment.
(37). A production method in accordance with either one of (35) and (36), wherein said step bringing Agrobacterium 6b protein or the modified body thereof in contact with the chromatin structure-producing material in the chromatin structure-producing environment inside a plant cell.

### BRIEF DESCRIPTION OF DRAWINGS

FIG. 1. The typical phenotype of AK-6b transgenic tobacco.

(A) Gross morphology of aerial parts of tobacco plants. Plants were soil-grown in a greenhouse. (a) A tobacco plant transformed with empty vector pBI121, 14 days old. (b) A transgenic tobacco plant that had been transformed with the P35S-AK-6b gene, 14 days old. (c) A tobacco plant transformed with empty vector pBI121, 2 months old. (d) An AK-6b transgenic tobacco plant that exhibited a mild phenotype, 2 months old. (e) An AK-6b transgenic tobacco plant that exhibited a severe phenotype, 2 months old. Scale bars: 1 cm.
(B) Gross morphology of aerial parts of Arabidopsis plants. Plants were soil-grown in a covered container and the cover was removed 3 days after vernalization (DAV). (a) An AK-6b transgenic Arabidopsis plant, 10 DAV. (b) A non-transgenic Arabidopsis plant, 10 DAV. (c) An AK-6b transgenic Arabidopsis plant, 41 DAV. (d) A non-transgenic Arabidopsis plant, 41 DAV. Scale bars: 1 mm (a, b): and 1 cm (c, d).

FIG. 2. Anatomical analyses with cross sections from leaves and petioles.

(A) Cross sections of leaf blades. Sections were made from leaves with 5-6 cm in length of 45-day-old tobacco plants. (a) Section of a plant transformed with empty vector pBI121. (b) Section of an AK-6b transgenic tobacco plant that exhibited a mild phenotype. (c, d) Magnified views of the boxed areas in (a) and (b), respectively. Scale bars: 1 mm (a, b): and 0.3 mm (c, d). The asterisk in (d) indicates additional layers of small cells at the abaxial side.
(B) Cross sections of petioles. (a) Young leaf of a tobacco plant transformed with empty vector pBI121 and (e) a section made from the site indicated by the arrow. (b) Young leaf of an AK-6b transgenic tobacco plant with a mild phenotype and (f) a section made from the site indicated. (c and d) Two examples of young leaves from an AK-6b transgenic tobacco plant that exhibited severe phenotypes and (g and h) sections made from the sites indicated by arrows. Right panels (i, j, k, and 1) show magnified views of boxed regions in respective panels (e, f, g, and h). Scale bars: 1 mm (a, b, c, d): 0.3 mm (e, f, g, h): and 0.2 mm (i, j, k, l).

FIG. 3. Ectopic accumulation of transcripts of meristem- and cell-division-related genes in leaves of AK-6b transgenic tobacco plants.

(A) Northern blot analysis. Tobacco plants were grown as described in Materials and Methods. Poly(A)+ RNAs were isolated from mature leaves with lengths of 5-6 cm of non-transgenic tobacco (lane 1), shoot apices of non-transgenic tobacco (lane 2), and mature leaves with lengths of 5-6 cm of AK-6b transgenic tobacco exhibiting the mild phenotype (lane 3) and the severe phenotype (lane 4). Tobacco genes examined are indicated at left.
(B) In situ hybridization of transcripts of the NACK1 gene for M-phase-specific kinesin-like protein. All the sections were made from the shoot apex and leaves with lengths of 5-6 cm of tobacco plants grown as described in (A). (a) Region of the shoot apical-meristem of a non-transgenic tobacco. (b) Leaf section containing the midvein and (c) a leaf blade of an AK-6b transgenic tobacco plant that exhibited a mild phenotype. (d) Leaf of an AK-6b transgenic tobacco plant with a mild phenotype probed with a sense probe. Scale bars: 0.1 mm (a): 0.3 mm (b): 0.2 mm (c): and 0.3 mm (d).

FIG. 4. Ectopic accumulation of transcripts of meristem- and cell-division-related genes in leaves of AK-6b transgenic Arabidopsis plants. Poly(A)+ RNAs were isolated from the first and the second leaves of 13-day-old wild-type (Col-0) and AK-6b transgenic plants. Amounts of transcripts of indicated genes were quantified by Real-time PCR as described in Materials and Methods. Each value was normalized to that of transcripts of the EF1-αgene. Relative values were calculated by dividing the values from 6b transgenic plants by the values from wild-type plants. FIG. 5. Effects of the nuclear import of AK-6b-GR on leaf morphology of transgenic tobacco.

(A) Plantlets of tobacco transformed with empty vector pSK1 (a and b), and the P35S-linked AK-6b::GR construct (c and d). Seeds were germinated on the medium supplemented without DEX (a and c) or with DEX (10 μM for b and d) and plants were grown for 16 d.
(B) Magnified views of leaves of AK-6b::GR transgenic tobacco grown in the medium without DEX (c) or with DEX (d). Scale bars: 2 mm.
(C) Subcellular localizations of sGFP-AK-6b-GR proteins in BY-2 cells incubated without DEX (a) or with DEX (10 μM for b). Scale bars: 0.01 mm.
(D) Intensities of fluorescence due to sGFP-AK-6b-GR in the cells treated without DEX (a) or with DEX (b) were measured by using the NIH ImageJ software (http://rsb.info.nih.gov/ij/). Graphs show three-dimensional histograms of the fluorescent intensities of pixels in pseudo color image. Numbers on the right indicate relative intensities.

FIG. 6. Effects of the nuclear import of sGFP-AK-6b-GR on callus formation from leaf sections of transgenic tobacco.

(A) Leaf sections of transgenic tobacco plants transformed with the P35S-linked sGFP construct (a and b), P35S-linked sGFP::AK-6b (c and d), and P35S-linked sGFP::AK-6b::GR (e and f) were incubated for 35 d on the medium supplemented without DEX (a, c, and e) or with DEX (10 μM for b, d, and f). The pictures on the right in each experiment are magnified views of the sections circled.
(B) Quantitative examination of callus formation by sGFP::AK-6b and sGFP::AK-6b::GR. One hundred leaf sections were examined for callus formation in each test as described in (A).

Numbers of sections that produced calli smaller than 1 mm, and those larger than 1 mm, were counted. Data are presented as percentages.

FIG. 7. Mitogenic activity of 6b and its interaction with histone H3. a, Schematic representation of the structural organization of 6b and its derivatives. b, Callus formation assay. c, Characterization of proteins used in the callus-formation assay (Scale bar=5 mm). d, Binding of 6b and its derivatives to histone H3. e, Binding of 6b to the histone fold. His-T7-tagged deleted variants of histone H3.2 (His-T7-H3.2) and His-tagged 6b (His-6b), as indicated below the photograph, were incubated together and complexes were precipitated with T7-specific antibodies. Recovered complexes were immunoblotted with His-specific antibodies. Arrowheads indicate positions of various truncated derivatives of histone H3.2.

FIG. 8. Interaction of 6b with histone H3 in tobacco cells. a, Detection of 6b in the chromatin fraction. Silver staining (left) and immunoblotting (right) analyses with T7-(top), histone H3-(middle) and catalase-(bottom) specific antibodies. b, Association of 6b with histone H3. Immunoprecipitates (IP) were recovered from the chromatin fraction with T7-specific antibodies and immunoblotted with His-(left) and histone H3-(right) specific antibodies. c, Release of 6b from chromatin by NaCl. Nuclei (Nt) were treated with indicated concentrations of NaCl. Pellets (P) were separated from supernatants (S) and equivalent amounts of protein were subjected to silver staining (top) and immunoblotting with T7-(middle) and histone H3-(bottom) specific antibodies.

FIG. 9. Histone-chaperone activity of 6b in vitro. a, The ability of human NAP-1 (lanes 5-8) and 6b (lanes 9-12) to introduce supercoils into relaxed plasmid DNA in the presence of core histones. b, Stoichiometry of the levels of 6b and core histones in the supercoiling reaction. c, Supercoiling activities of the AB-6b protein and mutant derivatives of AK-6b. d. Detection of the nucleosomal arrays assembled by human NAP-1 (lanes 5-8) and 6b (lanes 9-12) by partial digestion with MNase. R, relaxed plasmid DNA; S, supercoiled DNA; Mono and Di, lengths (bp) corresponding to polynucleotides covered with mono- and dinucleosomes, respectively.

### BEST MODE FOR CARRYING OUT THE INVENTION

Gene recombination techniques (including recombinant DNA techniques) as can be used herein include those known in the art, and can be carried out by the methods described in, for example, J. Sambrook, E. F. Fritsch & T. Maniatis, “Molecular Cloning: A Laboratory Manual (2nd edition)”, Cold Spring Harbor Laboratory Press, Cold Spring-Harbor, N.Y. (1989); D. M. Glover et al. ed., “DNA Cloning”, 2nd ed., Vol. 1 to 4, (The Practical Approach Series), IRL Press, Oxford University Press (1995); The Japanese Biochemical Society (JBS) ed., “Zoku-Seikagaku Jikken Koza 1, Idenshi Kenkyu-Hou II”, Tokyo Kagaku Dozin Co. Ltd., Japan, (1986); JBS ed., “Shin-Seikagaku Jikken Koza 2, Kakusan III (Recombinant DNA technique)”, Tokyo Kagaku Dozin Co. Ltd., Japan, (1992); “Methods in Enzymology” series, Academic Press, New York, including, for example, R. Wu ed., “Methods in Enzymology”, Vol. 68 (Recombinant DNA), Academic Press, New York (1980); R. Wu et al. ed., “Methods in Enzymology”, Vol. 100 (Recombinant DNA, Part B) & 101 (Recombinant DNA, Part C), Academic Press, New York (1983); R. Wu et al. ed., “Methods in Enzymology”, Vol. 153 (Recombinant DNA, Part D), 154 (Recombinant DNA, Part E) & 155 (Recombinant DNA, Part F), Academic Press, New York (1987); J. H. Miller ed., “Methods in Enzymology”, Vol. 204, Academic Press, New York (1991); R. Wu ed., “Methods in Enzymology”, Vol. 216 (Recombinant DNA, Part G), Academic Press, New York (1992); R. Wu ed., “Methods in Enzymology”, Vol. 217 (Recombinant DNA, Part H) & 218 (Recombinant DNA, Part I), Academic Press, New York (1993); G. M. Attardi et al. ed., “Methods in Enzymology”, Vol. 260 (Mitochondrial Biogenesis and Genetics, Part A), Academic Press, New York (1995); J. L. Campbell ed., “Methods in Enzymology”, Vol. 262 (DNA Replication), Academic Press, New York (1995); G. M. Attardi et al. ed., “Methods in Enzymology”, Vol. 264 (Mitochondrial Biogenesis and Genetics, Part B), Academic Press, New York (1996); P. M. Conn ed., “Methods in Enzymology”, Vol. 302 (Green Fluorescent Protein), Academic Press, New York (1999); S. Weissman ed., “Methods in Enzymology”, Vol. 303 (cDNA Preparation and Characterization), Academic Press, New York (1999); J. C. Glorioso et al. ed., “Methods in Enzymology”, Vol. 306 (Expression of Recombinant Genes in Eukaryotic Systems), Academic Press, New York (1999); M. Ian Phillips ed., “Methods in Enzymology”, Vol. 313 (Antisense Technology, Part A: General Methods, Methods of Delivery and RNA Studies) & 314 (Antisense Technology, Part B: Applications), Academic Press, New York (1999); J. Thorner et al. ed., “Methods in Enzymology”, Vol. 326 (Applications of Chimeric Genes and Hybrid Proteins, Part A: Gene Expression and Protein Purification), 327 (Applications of Chimeric Genes and Hybrid Proteins, Part B: Cell Biology and Physiology) & 328 (Applications of Chimeric Genes and Hybrid Proteins, Part C: Protein-Protein Interactions and Genomics), Academic Press, New York (2000) etc., or by methods described in the references cited therein or methods substantially equivalent thereto or modified methods thereof, the disclosures of which are incorporated herein by reference.

Present invention relates to the modification of gene expression. More particularly, the present invention relates to method for modifying gene expression of plant body, genetically modified plant body, method of producing a genetically modified plant body, method of producing useful substances, modified plant chromatin structure and use thereof.

According to the present inventor findings, Agrobacterium 6b protein can interact with histone H3 protein. Consequently, expression of gene related to morphology of plant and phenotype of plant leaf etc. are changed. Moreover, the expression pattern of IAA gene family and TCP family are changed globally. That is, expression levels of many of these gene family members are changed when Agrobacterium 6b protein is produced in the plant cell. These results shows that Agrobacterium 6b protein has a rule of histone chaperon activity, thereby alters or remodels chromatin structure to become to transcript different genes with that Agrobacterium 6b protein is not existed.

### (Plant Body)

In the present invention, plant body includes any form derived from plant. Specifically, it includes plant cells, cultured plant cells, protoplasts, shoot primordia, multiple shoots, hairy roots callus, plant tissues, plant organs and plant individual bodies, breeding body (seed). In the present invention, the applicable plant is not limited to but selected from known plants, including for example cultivated plants, useful or valuable plants and other plants. Such plants include those known as cereal crops, beans, root and tuber crops, nuts, seeds, vegetables, berries, fruits, or orchard trees, further derived from garden crops, garden trees or grasses, horticultural species, flowering trees and ornamental species. Examples of the plant cells are those selected from the group consisting of Solanaceous species, Cruciferous (Brassicaceous) species, Graminaceous species, Leguminous species, Liliaceous species, Umbelliferous species, Cucurbitaceous species and other species, preferably tobacco (Nicotiana tabacum), Arabidopsis thaliana, soybean (Glycine max), Aduki bean (small red bean), green beans (Phaseolus vulgaris), peas (Lathyrus spp. including snow pea (Pisum sativum)), broad bean (Vicia faba), peanuts (Arachis hypogaea), sesame (Sesamum indicum LINNE), rice (Oryza sativa), wheat (Triticum aestivum), barley, rye (Secale cereale), oat (Avena sativa), corn (Zea mays), sorghum (Sorghum bicolor, Sorghum vulgare), potato (Solanum tuberosum), tomato, green pepper, cabbage, broccoli, parsley, spinach, sweet potato (Ipomoea batatus), taro (Colocasia spp. including C. esculenta), konjac (Amorphophallus rivieri var. konjac), cassaya (Manihot spp. including M. esculenta), grapes, apple, peach, pear (Pyrus spp. including Pyrus pyrifolia var. culta and Pyrus communis), persimmon (Diospyros kaki), strawberries, blueberries, plum, melon, cucumber, sugar cane (Saccharum officinarum L.), tangerine, lemon, orange, olive (Olea europaea), cotton (Gossypium arboretum) and other plant cells.

Preferably, plants are selected from Agrobacterium infectious plant. These plants may include Gymnosperms, Angiosperms, Dicotyledonae and Monocotyledonae. In the present invention plant are not limited to, but may be selected from any species including those widely known as cultivated plants or useful plants. The plants include cereal crops, beans, root and tuber crops, nuts, seeds, vegetables, berries, fruits, or orchard trees, as well as garden crops, garden trees or grasses, horticultural species, flowering trees and ornamental species. Exemplary plants include Solanaceous species, Cruciferous (Brassicaceous) species, Graminaceous species, Leguminous species, Liliaceous species, Umbelliferous species, Cucurbitaceous species and other species, preferably tobacco (Nicotiana tabacum), Arabidopsis thaliana, soybean (Glycine max), Aduki bean (small red bean), green beans (Phaseolus vulgaris), peas (Lathyrus spp. including snow pea (Pisum sativum)), broad bean (Vicia faba), peanuts (Arachis hypogaea), rice (Oryza sativa), wheat (Triticum aestivum), barley, rye (Secale cereale), oat (Avena sativa), bentgrass (Agrostis spp.), corn (Zea mays), sorghum (Sorghum bicolor, Sorghum vulgare), canola (Brassica napus, Brassica rapa ssp.), rape (Brassica napus), potato (Solanum tuberosum), sweet potato (Ipomoea batatus), taro (Colocasia spp. including C. esculenta), konjac (Amorphophallus rivieri var. konjac), cassaya (Manihot spp. including M. esculenta), and others.

### (Agrobacterium 6b Protein)

Present invention provides use of Agrobacterium 6b protein and modified body thereof based on its histone-chaperone-activity. Agrobacterium 6b proteins are conserved among Agrobacteriums. Therefore, Agrobacterium 6b proteins can be used as the protein having histone-chaperon-activity. In present invention for example ‘Agrobacterium’ includes Agrobacterium aurantiacum, Agrobacterium gelatinovorum, Agrobacterium radiobacter, Agrobacterium rhizogenes, Agrobacterium sp., Agrobacterium tumefaciens, Agrobacterium vitis and the like.

Histone-chaperone-activity of Agrobacterium 6b protein can be related to property of interacting with histone and forming nucleosome. Interacting region of 6b protein can comprises C-terminal side of the protein. The C-terminal region comprises acidic amino acid sequence and franking C-terminal sequence in the C-terminal end. These acidic sequences among Agrobacterium 6b proteins are well conserved also. The invention is not restricted by following presumption, but these C-terminal sequences have important rule for histone-chaperone-activity. For example, in the present invention Agrobacterium 6b protein shown in SEQ ID NO:2 can be used. In this sequence, the acidic sequence is 164 to 184 of the sequence. Also, proteins which are functionally equivalent to Agrobacterium 6b protein ca be used. As used herein, the term “equivalently functional” or “functionally equivalent” indicates that a protein acts or functions as a histone chaperon protein associating histone H3 protein. It is possible to assess whether or not proteins serve as histone chaperon protein associating histone H3 protein, through according to Example in the later part of the specification.

Modified body of Agrobacterium 6b protein means modified in histone chaperon activity. More particularly, modified proteins have different histone chaperon activity in quantity and quality from naturally occurring Agrobacterium 6b protein (for example, protein shown in SEQ ID NO:2). Such modified protein can be obtained by modification of the amino acid sequence of C-terminal acidic amino acid region and/or franking C-terminal end region thereof and the amino acid sequence other than said acidic amino acid region and said franking C-terminal region.

In an embodiment of methods for obtaining or isolating the aforementioned functionally equivalent proteins or modified proteins, techniques for incorporating an amino acid mutation into a protein are well known to persons skilled in the art. It is possible for a person skilled in the art to incorporate an amino acid substitution, deletion (disruption), and/or insertion (addition) into the amino acid sequence of a naturally occurring “Agrobacterium 6b protein” (e.g., the protein shown in SEQ ID NO: 2) according to a conventional technique to produce a mutated protein functionally equivalent to such a naturally occurring protein or modified protein. Amino acid mutations may also occur naturally. The proteins of the present invention include those having an amino acid sequence containing at least one substitution, deletion (disruption), or insertion (addition) of one or plural amino acid residues in the amino acid sequence of such a naturally occurring “Agrobacterium 6b protein”. The number of altered amino acid residues in sequence is usually 50 or less per total of amino acid residues present in a protein, preferably 30 or less, still preferably 10 or less and more preferably 3 or less. Amino acid modifications can be performed using commercially available kits.

Such mutations, conversions and modifications include those described in JBS (Ed.), “Zoku-Seikagaku Jikken Koza 1, Idenshi Kenkyuhou II”, p 105 (Susumu Hirose), Tokyo Kagaku Dozin Co. Ltd., Japan (1986); JBS (Ed.), “Shin-Seikagaku Jikken Koza 2, Kakusan III (Recombinant DNA technique)”, p 233 (Susumu Hirose), Tokyo Kagaku Dozin Co. Ltd., Japan (1992); R. Wu, L. Grossman, ed., “Methods in Enzymology”, Vol. 154, p. 350 & p. 367, Academic Press, New York (1987); R. Wu, L. Grossman, ed., “Methods in Enzymology”, Vol. 100, p. 457 & p. 468, Academic Press, New York (1983); J. A. Wells et al., Gene, 34: 315, 1985; T. Grundstroem et al., Nucleic Acids Res., 13: 3305, 1985; J. Taylor et al., Nucleic Acids Res., 13: 8765, 1985; R. Wu ed., “Methods in Enzymology”, Vol. 155, p. 568, Academic Press, New York (1987); A. R. Oliphant et al., Gene, 44: 177, 1986, etc. For example, included are methods such as site-directed mutagenesis (site-specific mutagenesis) utilizing synthetic oligonucleotides (Zoller et al., Nucl. Acids Res., 10: 6487, 1987; Carter et al., Nucl. Acids Res., 13: 4331, 1986), cassette mutagenesis (Wells et al., Gene, 34: 315, 1985), restriction selection mutagenesis (Wells et al., Philos. Trans. R. Soc. London Ser A, 317: 415, 1986), alanine scanning (Cunningham & Wells, Science, 244: 1081-1085, 1989), PCR mutagenesis, the Kunkel method, dNTP[.alpha.S] method (Eckstein), region directed mutagenesis using sulfurous acid and nitrous acid and the like.

Such proteins of the present invention can be produced as recombinant proteins which are prepared using gene recombination techniques. The recombinant proteins can be prepared by conventional methods, for example by insertion of the inventive protein-coding DNA into a suitable expression vector, introduction of said vector construct into a suitable cell, and purification from the resultant transformed cell.

In addition other embodiments of functionally equivalent protein isolation include hybridization techniques (Southern, J. Mol. Biol. 98: 503 (1975); Maniatis et al., “Molecular Cloning”, Cold Spring harbor Laboratory Press) and PCR techniques (H. A. Erlich (ed.), “PCR technology”, Stockton Press, New York (1989)). In other words, it is possible for a person skilled in the art to obtain proteins functionally equivalent to the Agrobacterium 6b protein via using isolated DNA highly homologous to the nucleotide sequence of the Agrobacterium 6b gene (SEQ ID NO: 1) wherein said isolated DNA is obtained using the nucleotide sequence of the Agrobacterium 6b protein gene (SEQ ID NO: 1) or part thereof as a probe as well as using oligonucleotides hybridizable with part of the nucleotide sequence of the Agrobacterium 6b protein gene (SEQ ID NO: 1) as primers. In addition, person skilled in the Art can obtain a plant body producing these proteins.

### (Histone Protein)

In the present invention, histone protein is a target protein of Agrobacterium 6b protein for altering or remodeling of plant chromatin structure. In the present invention, histone protein can be histone H3. Agrobacterium 6b protein can interact with region of histone H 3.2, particularly, folding domain containing arfa1, arufa2 and arufa3. Instead of endogenous histone protein in the present invention using modified body of histone protein with modification in folding domain and/or non-fording domain allow to alter plant chromatin structure and modify gene expression compared with that of using naturally occurring histone. Such modified protein can be obtained by well-known techniques in person skilled in the Art as described above. Also person skilled in the Art can obtain a plant body producing such proteins.

### (Method for Genetically Modifying Plant Body and Modifying Gene Expression of Plant Body)

In the present invention, a first modification of plant body is to introduce a gene coding Agrobacterium 6b protein or functionally equivalent protein thereof and allow expressing the gene. According to the modification, chromatin structure can be altered or remodeled by interaction between Agrobacterium 6b protein and histone and changed (enhanced and/or repressed) expression pattern of a specific gene groups globally. For example, Agrobacterium 6b protein can change one or more, preferably two or more genes selected from IAA gene family members and TCP family members.

Typical gene members are listed in Table 1 and table 2

TABLE 1

| | | Ratio | Ratio Real- | Auxin |
|---|---|---|---|---|
| IAA | | microarray | time PCR | induc- |
| genes | gene ID | 6b/WT * | 6b/WT § | tivit ¶ |
| IAA1 | AT4G14560.1 | 0.47 (0.44; 0.51) | 0.28 ± 0.02 | + |
| IAA2 | AT3G23030.1 | 0.47 (0.43; 0.51) | 0.42 ± 0.06 | + |
| IAA3/ | AT1G04240.1 | 0.25 (0.24; 0.25) | 0.25 ± 0.03 | + |
| SHY2 | | | | |
| IAA4 | AT5G43700.1 | 0.46 (0.44; 0.48) | 0.24 ± 0.08 | + |
| IAA5 | AT1G15580.1 | 0.87 (0.85; 0.90) | 0.29 ± 0.09 | + |
| IAA6 | AT1G52830.1 | 0.46 (0.45; 0.46) | 0.01 ± 0.01 | + |
| IAA7/ | AT3G23050.1 | 0.66 (0.66; 0.67) | 0.61 ± 0.08 | + |
| AXR2 | | | | |
| IAA8 | AT2G22670.1 | 0.56 (0.54; 0.59) | 0.47 ± 0.08 | − |
| IAA9 | AT5G65670.1 | 0.81 (0.72; 0.91) | 0.71 ± 0.08 | + |
| IAA10 | AT1G04100.1 | 1.16 (1.07; 1.25) | 0.82 ± 0.15 | + |
| IAA11 | AT4G28640.1 | 0.67 (0.65; 0.68) | 0.55 ± 0.24 | + |
| IAA12/ | AT1G04550.1 | 0.96 (0.95; 0.96) | 1.03 ± 0.12 | − |
| BDL | | | | |
| IAA13 | AT2G33310.1 | 0.81 (0.71; 0.90) | 1.27 ± 0.15 | + |
| IAA14/ | AT4G14550.1 | 0.49 (0.49; 0.50) | 0.51 ± 0.02 | + |
| SLR1 | | | | |
| IAA15 | AT1G80390.1 | 1.04 (0.87; 1.20) | 0.92 ± 0.62 | N.D |
| IAA16 | AT3G04730.1 | 0.53 (0.47; 0.60) | 0.56 ± 0.09 | + |
| IAA17/ | AT1G04250.1 | 0.27 (0.27; 0.27) | 0.24 ± 0.04 | + |
| AXR3 | | | | |
| IAA18 | AT1G51950.1 | 0.96 (0.90; 1.03) | 0.97 ± 0.16 | − |
| IAA19/ | AT3G15540.1 | 0.72 (0.66; 0.79) | 0.65 ± 0.05 | + |
| MSG2 | | | | |
| IAA20 | AT2G46990.1 | 1.18 (1.18; 1.18) | 0.49 ± 0.08 | + |
| IAA26/ | AT3G16500.1 | 0.96 (0.79; 1.12) | 0.95 ± 0.06 | N.D |
| PAP1 | | | | |
| IAA27/ | AT4G29080.1 | 0.59 (0.53; 0.66) | 1.06 ± 0.12 | − |
| PAP2 | | | | |
| IAA28 | AT5G25890.1 | 1.20 (1.02; 1.36) | 0.74 ± 0.09 | − |
| IAA29 | AT4G32280.1 | 0.67 (0.63; 0.72) | 0.36 ± 0.01 | + |
| IAA30 | AT3G62100.1 | 0.62 (0.61; 0.63) | 0.34 ± 0.09 | + |
| IAA31 | AT3G17600.1 | 5.20 (5.08; 5.33) | 0.22 ± 0.04 | − |
| IAA32 | AT2G01200.1 | 0.66 (0.59; 0.73) | 0.16 ± 0.08 | N.D |
| IAA34 | AT1G15050.1 | 0.46 (0.45; 0.48) | 0.75 ± 0.21 | + |

Change in the expression level of IAA genes in 6b-gene introduced Arabidopsis plant.
Ratio of the expression level of each IAA gene and the inductivity by Arabidopsis in 6b-gene introduced Arabidopsis plant to that in wild type Arabidopsis plant is shown.
* The average value obtained by 2 independent tests, each value of the tests are shown in parenthesis.
§ The average value of tests performed at least 4 times.
¶ Referring to the microarray tests(TAIR database, submission number: ME00336) performed in the past,
+ represents a expression level increase by 1.5 or more times by Arabidopsis,
− represents no increase.
N.D: not contained in the database of the microarray.

TABLE 2

| | | Ratio microarray | Ratio Real-time PCR |
|---|---|---|---|
| TCP genes | gene ID | 6b/WT * | 6b/WT ¶ |
| TCP2 | AT4G18390.1 | 1.13 (1.04; 1.21) | |
| TCP3 | AT1G53230.1 | 1.02 (0.99; 1.06) | |
| TCP4 | AT3G15030.1 | 1.53 (1.42; 1.64) | 11.5 ± 1.94 |
| TCP5 | AT5G60970.1 | 2.03 (1.76; 2.29) | 1.80 ± 0.15 |
| TCP8 | AT1G58100.1 | 1.36 (1.33; 1.39) | 1.07 ± 0.16 |
| TCP9 | AT2G45680.1 | 1.21 (1.09; 1.33) | |
| TCP10 | AT2G31070.1 | 1.89 (1.83; 1.96) | 2.08 ± 0.2 |
| TCP11 | AT2G37000.1 | N.D | N.D |
| TCP13 | AT3G02150.1 | 1.64 (1.52; 1.77) | 1.01 ± 0.27 |
| TCP14 | AT3G47620.1 | 1.03 (1.00; 1.06) | |
| TCP15 | AT1G69690.1 | 1.00 (0.97; 1.03) | |
| TCP17 | AT5G08070.1 | 1.05 (0.99; 1.11) | |
| TCP19 | AT5G51910.1 | 0.91 (0.88; 0.94) | |
| TCP20 | AT3G27010.1 | 2.07 (1.95; 2.19) | 1.66 ± 0.24 |
| TCP21 | AT5G08330.1 | 0.92 (0.86; 0.98) | |
| TCP23 | AT1G35560.1 | 1.26 (1.19; 1.32) | |
| TCP24 | AT1G30210.1 | 1.24 (1.17; 1.32) | |

Change in the expression level of TCP genes in 6b-gene introduced Arabidopsis plant.
Ratio of the expression level of each TCP gene in 6b-gene introduced Arabidopsis plant to that in wild type Arabidopsis plant is shown.
* The average value obtained by 2 independent tests, each value of the tests are shown in parenthesis.
¶ The average value of tests performed at least 4 times.
N.D.: No gene chip recognizing TCP11 was observed on the array. And accumulated transfer product was undetectable in Real-time PCR.

As shown in these Tables, in IAA gene family members most of the members are repressed. Particularly, IAA1, IAA2, IAA3/SHY2, IAA4, IAA6, IAA8, IAA14/SLR1, IAA16, IAA17/AXR3, IAA27/PAP2, IAA30 and IAA34 are repressed about 50 percent. More Particularly, IAA1, IAA2, IAA3, IAA6, IAA16, IAA17 and IAA34 are repressed. Further IAA3 and IAA17 are strongly repressed. The other hand, in TCP gene family members most of the members are increased or not changed but no member is repressed. Particularly, TCP4, TCP5, TCP10, TCP13 and TCP20 are increased. More particularly, TCP5, TCP10 and TCP20 are increased.

In addition to IAA gene family members and TCP gene family members, KNOX genes (STM, BP, KNAT2 and KNAT6), CUC1, CUC2, CUC3, AtNAK1/HK and cyclin B are increased. Particularly, STM, CUC2 and CUC 3 are strongly increased.

A second modification of plant body of the present invention is to introduce a gene allowing repress expression of Agrobacterium 6b protein for particularly Agrobacterium infected plant body. According to the modification, chromatin structure can be altered or remodeled by suppress interaction between Agrobacterium 6b protein and histone and changed expression pattern of a specific gene groups globally. For example, expression of one or more, preferably two or more genes selected from above genes including IAA gene family members, TCP family members and KNOX genes and the like can be changed.

A third modification of plant body of the present invention is to introduce a gene coding Agrobacterium 6b protein or functionally equivalent protein thereof and a gene coding endogenous histone or exogenous (it can be a modified histone). According to the modification, chromatin structure can be altered or remodeled based on alteration of interaction between Agrobacterium 6b protein and histone and changed expression pattern of a specific gene groups globally. For example, expression of one or more, preferably two or more genes selected from IAA gene family members, TCP family members and KNOX genes and the like can be changed.

A forth modification of plant body of the present invention is to introduce a gene coding modified body of Agrobacterium 6b protein. According to the modification, chromatin structure can be altered or remodeled based on alteration of interaction between modified body of Agrobacterium 6b protein and histone and changed expression pattern of a specific gene groups globally. For example, expression of one or more, preferably two or more genes selected from IAA gene family members, TCP family members and KNOX genes and the like can be changed.

In these modifications one or two more genes coding protein of interest can be introduced into plant body. This modification can allow expressing one or more gene of interest and functioning in plant body under modification by Agrobacterium 6b protein.

To modify plant body genetically, well-known genetically recombination techniques in person skilled in the Art can be used. Person skilled in the Art can easily select appropriate materials and methods and perform the modification of plant in the present invention. Applicable incorporation of a vector or vector construct into a plant body includes, for example, Agrobacterium-mediated transformation (Hood et al., Transgenic Res., 2: 218 (1993); Hiei et al., Plant J., 6: 271 (1994)), electroporation (Tada et al., Theor. Appl. Genet, 80: 475 (1990)), polyethylene glycol method (Lazzeri et al., Theor. Appl. Genet, 81: 437 (1991)), particle bombardment (Sanford et al., J. Part. Sci. tech., 5: 27 (1987)). The incorporation (introduction) technique is suitably selected those known in the art and well described in the scientific and patent literature. In addition, for repressing gene expression for Agrobacterium 6b protein and the like, anitisence technique, ribozyme technique, co-suppression, RNA interference, technique of introducing dominant negative gene

### (DNA Coding Proteins)

The present invention also provides DNA coding for each of the aforementioned proteins according to present invention. The DNA of the present invention is not limited to but includes genomic DNA, cDNA, chemically synthesized DNA, and others as long as it is capable of encoding the protein of the present invention. Genomic DNA can be prepared, for example by conducting a polymerase chain reaction (PCR) wherein template genome DNA prepared according to the method disclosed in the document (Rogers and Bendich, Plant Mol. Biol. 5: 69 (1985)) is used in combination with primers constructed on the basis of the DNA sequences (e.g., the nucleotide sequence of SEQ ID NO: 31) of the present invention. For cDNA, mRNA prepared from plants according to conventional techniques (Maniatis et al. Molecular Cloning Cold Spring harbor Laboratory Press) is subjected to reverse transcription followed by PCR using the aforementioned primers to prepare the cDNA. Also, genomic DNA and cDNA can be prepared by production of genomic DNA libraries or cDNA libraries according to conventional techniques, and screening for the resultant genomic DNA libraries or cDNA libraries with probes synthesized on the bases of the nucleotide sequence of the present invention (e.g., the nucleotide sequence of SEQ ID NO: 1).

It is convenient to isolate DNAs coding for plant proteins aforementioned relying on hybridization and methods relying on PCR. Utilizable plants in the present invention which Agrobacterium 6b protein is isolated or separated can be selected from Dicotyledonae plants. These plants can also be used as gene sources.

The present invention still provides recombinant DNAs and vectors (vector constructs) comprising an insert of DNA which suppresses or inhibits the aforementioned DNA of the present invention, expression of the DNA genes of the present invention. The recombinant DNAs and vectors (vector constructs) include, besides the aforementioned vectors (vector constructs) utilized in production of recombinant proteins, vectors or vector constructs to allow expression of DNAs in plant cells for production of transgenic plants, said DNAs which suppress or inhibit the DNA of the present invention, expression of the DNA of the present invention, and the expression of proteins encoded by the DNA of the present invention. Such recombinant DNAs or vectors (vector constructs) are not limited to, but include any as long as they comprise a promoter sequence transcribable in plant cells and a terminator sequence containing a polyadenylation site required for stabilization of transcript products. Examples of the recombinant DNAs or vectors (vector constructs) are plasmids, “pBI121”, “pBI221”, “pBI101” (all, Clontech), “pTA7001”, “pTA7002” (Aoyama et al. (1997) Plant J. 11: 605), “pPZP211” (Hajdukiewicz et al., Plant Mol. Biol. 25: 989 (1994), etc.

The aforementioned recombinant DNA or vector (vector construct) of the present invention may comprise any of promoters designed to allow stable or inducible expression of the proteins according to the present invention. The promoters expressible in vivo herein are desirably those as listed herein below. Examples of promoters designed to allow stable expression are the cauliflower mosaic virus (CaMV) 35S promoter (Odell et al., Nature, 313: 810 (1985)), the rice actin promoter (Zhang et al., Plant Cell, 3: 1155 (1991)), the maize ubiquitin promoter (Cornejo et al., Plant Mol. Biol., 23: 567 (1993)), etc.

Promoters designed to allow inducible expression include, for example, those known as elements which are expressible by exogenous factors including filamentous fungal, bacterial, or viral infection or invasion, low temperatures, elevated temperatures, dry conditions, UV light radiation, and applications of specific compounds. Examples of such promoters are those for expression induced by filamentous fungal, bacterial, or viral infection or invasion, such as the rice chitinase gene promoter (Xu et al., Plant Mol. Biol., 30: 387 (1996)) and the tobacco PR protein gene promoter (Ohshima et al., Plant Cell 2: 95 (1990)); the rice “lip19” gene promoter inducible by low temperatures (Aguan et al., Mol. Gen. Genet., 240: 1 (1993)); the rice “hsp80” gene or “hsp72” gene promoter inducible by high temperatures (Van Breusegem et al., Planta, 193: 57 (1994)); the Arabidopsis thaliana “rab16” gene promoter inducible by desiccation (Nundy et al., Proc. Natl. Acad. Sci. USA, 87: 1406 (1990)); the parsley chalcone synthase gene promoter inducible by UV light radiation (Schulze-Lefert et al., EMBO J., 8: 651 (1989)); the maize alcohol dehydrogenase gene promoter inducible by anaerobic conditions (Walker et al., Proc. Natl. Acad. Sci. USA, 84: 6624 (1987)); etc. Also, the rice chitinase gene promoter and the tobacco PR protein gene promoter can be induced by specific compounds such as salicylic acid. The “rab16” can also be induced by applications of phytohormone abscisic acid. Included is use of vector systems comprising glucocorticoid- or estrogen-responsive systems to allow inducible expression of genes in plants. The glucocorticoid-responsive vectors to allow inducible expression include pTA7001, pTA7002 (Aoyama et al., Plant J., 11: 605 (1997)). The estrogen-responsive vectors to allow inducible expression include PER10 (Zuo et al., Plant J., 24: 265 (2000)). Also, examples of the promoters to allow growing cell-specific expression of genes in plants are the tobacco NPK1 gene promoter expressible during S to M phases (Nishihama et al., Genes Dev., 15: 352 (2000)), the tobacco NACK1 gene promoter as a promoter expressible at M phase (Nishihama et al., Cell, 109: 87 (2002)), the Catharanthus roseus CYM gene promoter (Ito et al., Plant J., 11: 983 (1997)), the Catharanthus roseus CYS gene promoter expressible at S phase (Ito et al., Plant J., 11: 983 (1997)), the Arabidopsis thaliana cdc2a gene promoter which is observed to be active in growing cells throughout cell cycle (Chung et al., FEBS Lett., 362: 215 (1995)), etc. Examples of the tissue-specific promoters can be the Arabidopsis thaliana AtHB8 promoter which acts as a vascular bundle procambium-specific promoter (Baima et al. Development 121: 4171 (1995)), the stem- or root-specific Arabidopsis thaliana ACL5 promoter (Hanzawa et al. The EMBO Journal, 19: 4248 (2000)), the terrestrial body-specific tomato RBCS3A promoter (Meier et al. Plant Physiol. 107: 1105 (1995)), etc.

The promoters which are active at high gene expression levels in the male reproductive organs or cells, include the Arabidopsis thaliana AtNACK2 gene promoter (PCT/JP02/12268), the Arabidopsis thaliana AVP1 gene promoter (Mitsuda et al., Plant Mol. Biol, 46: 185 (2001)), the Arabidopsis thaliana DAD 1 gene promoter (Ishiguro et al., Plant Cell, 13: 2191 (2001)), the tobacco TA20 & TA29 gene promoters (Goldberg et al., Science, 240: 1460 (1988)), the rice Osg6B gene promoter (Tuchiya et al., Plant Mol. Biol, 26: 1737 (1994)), the tomato Lat52 gene promoter (Twellr et al., Development, 109: 705 (1990)), the tobacco g10 gene promoter (Rogers et al., Plant Mol. Biol., 45: 577 (2001)), and artificial promoters derived by insertion of an another-specific regulatory sequence for gene expression into the cauliflower mosaic virus 35S promoter (Ingrid et al., Plant Cell, 4: 253 (1992)), etc.

Also, the present invention provides transformed plant body into which the aforementioned recombinant DNA or vector (or vector construct) of the present invention is incorporated. The plant body into which the vector (or vector construct) of the present invention is incorporated includes those plant cells for giving transformed plant bodies (plant transformants). The plant body into which the vector or vector construct of the present invention is transferred includes plant cells for producing or constructing transgenic plants or plant bodies. Applicable incorporation of a vector or vector construct into a plant cell includes, for example, Agrobacterium-mediated transformation (Hood et al., Transgenic Res., 2: 218 (1993); Hiei et al., Plant J., 6: 271 (1994)), electroporation (Tada et al., Theor. Appl. Genet, 80: 475 (1990)), polyethylene glycol method (Lazzeri et al., Theor. Appl. Genet, 81: 437 (1991)), particle bombardment (Sanford et al., J. Part. Sci. tech., 5: 27 (1987)). The incorporation (introduction) technique is suitably selected those known in the art and well described in the scientific and patent literature.

### (Modified Plant Body)

Transformed plant cells can be redifferentiated to regenerate plant bodies. Redifferentiation varies depending on plant cell species, but includes, for example, Fujimura et al. method (Plant Tissue Culture Lett., 2: 74 (1995)) for rice; Shillito et al. method (Bio/Technology, 7: 581 (1989)) and Gorden-Kamm et al. method (Plant Cell, 2: 603 (1990)) for corn; Visser et al. method (Theor. Appl. Genet, 78: 594 (1989)) for potato; Nagata & Takebe method (Planta, 99: 12 (1971)) for tobacco; and Akama et al. method (Plant Cell Reports, 12: 7-11 (1992)) for Arabidopsis thaliana.

Once transgenic plants are produced into which inhibitory or suppressive DNA is incorporated against the DNA of the present invention or expression of the inventive DNA, it is possible to obtain off-springs from the plants via sexual or asexual (vegetative) reproduction. It is possible to propagate plants in a mass scale on the basis of propagation materials (for example, seeds, nuts, fruits, cuttings, tubers, rhizomes, bulbs, shoots, roots, offsets, plant calli, protoplasts, etc.) obtained from the plant of interest, and off-springs or clones thereof. The present invention encompasses plant cells into which suppressive DNA has been incorporated against the DNA of the present invention or expression of the inventive DNA, plants harboring the same plant cell, off-springs and clones derived from the same plant, said plants or plant bodies, their propagation materials (sources) such as off-springs and clones.

### (Method for Producing Modified Plant Body and Useful Substances)

The present invention further provides method for producing modified plant body and method for producing useful substances. According these methods because of modified or altered gene expression pattern, particularly, globally modified or altered gene expression pattern. Further, modified or altered gene expression pattern can be stably maintained in plant body. For this reason useful substances can be produced continuously and modified plant itself can be proliferated stably. For producing a useful substance it is preferred to use plant cell (cultured plant cell), tissue culture and callus in case that modified plant body carries serious aberration.

The present invention further provides method for producing modified plant body and method for producing useful substances. According these methods because of modified or altered gene expression pattern, particularly, globally modified or altered gene expression pattern. Further, modified or altered gene expression pattern can be stably maintained in plant body. For this reason useful substances can be produced continuously and modified plant itself can be proliferated stably. For producing a useful substance it is preferred to use plant cell (cultured plant cell), tissue culture and callus in case that modified plant body carries serious aberration.

### (Chromatin Structure)

The present invention further provides modified chromatin structure carrying Agrobacterium 6b protein. According the chromatin structure can modify and alter gene expression pattern, particularly, globally. Further, modified or altered gene expression pattern can be stably maintained in plant body. Therefore the chromatin structure is useful to stable and continuous expression of genes.

### (Material and Methods)

Followings are applied to examples 1 to 3.

### Plant Materials

Tobacco (Nicotiana tabacum cv SR1) and Arabidopsis thaliana ecotype Columbia (Col-0) were used as the wild-type plants. Wild-type and AK-6b transgenic plants were grown as previously described (Semiarti et al. 2001; Kitakura et al. 2002). For analyses of gross morphology of tobacco, plants were grown on soil in a green house (light for 16 h and darkness for 8 h) at 28° C. For anatomical analyses and RNA preparation, tobacco plants were grown on Murashige and Skoog medium prepared with 1% agar in light for 16 h and in darkness for 8 h at 26° C. For analyses of phenotypes of Arabidopsis, seeds were sown on soil, and after 2 d at 4° C. in darkness, plants were transferred to a regimen of white light for 16 h and darkness for 8 h at 22° C. For RNA preparation from Arabidopsis plants, seeds were shown on Murashige and Skoog medium prepared with 1% agar and the plants were germinated and grown under conditions similar to those described above.

### Plasmid Constructs

Plasmid DNAs were constructed using PCR amplification and standard cloning techniques. The AK-6b gene from pTiAKE10 (Wabiko and Minemura 1996) was linked to the 35S promoter of Cauliflower mosaic virus (P35S) in the binary vector pBI121. The glucocorticoid receptor (GR) gene was fused to the 3′ end of sGFP-AK-6b of sGFP-AK-6b (Kitakura et al. 2002), which was linked to P35S in the binary vector pSK1 (Kojima et al. 1999).

### Analyses of Gene Expression

For analyses of gene expression of tobacco, plants were grown for 45 d as described above, leaves with lengths of 5-6 cm were isolated from the wild-type and AK-6b transgenic plants and RNA was prepared (Hamada et al. 2000). Arabidopsis plants were grown for 13 d after vernalization and RNA was prepared from the first and the second leaves. RNA gel blot analyses were performed as described elsewhere (Hamada et al. 2000) with the exception that total RNA from leaves of AK-6b transgenic tobacco and SR1 was prepared and the polyadenylated RNA (0.5 μg) was used.

To prepare a NTH15 probe, a 465 bp fragment, corresponding to the 5′ portion of NTH15 cDNA, was generated by BamHI and SacI cleavage of a plasmid that contained NTH15 cDNA. We used coding regions of CycD3; 1 (AJ011893) cDNA and AK-6b DNA for hybridization probes by cleavage of plasmids that contained CycD3; 1 cDNA (pNU449) and AK-6b DNA (pNU309). PCR fragments of NTH1, NTH20, NTH22, NACK1 and cyclinB cDNA were used to generate hybridization probes. We used primers specific for NTH1

(NTH1-1, 5′-GACCTGTTTCTCTCCCTCTTTA-3′ and NTH1-2, 5′-ATTGATGCCATTTCTTGGGGTG-3′), NTH20 (NTH20-1, 5′-GGTAATTAATGGAGAATAATTA-3′ and NTH20-2, 5′-GTACTGCCGATAGCCTCGCCAC-3′), NTH22 (NTH22-1, 5′-GATTATTTCTTTACTAATTCAC-3′ and NTH22-2, 5′-TGCTATCTCTGGTGGTGCTCCT-3′), NACK1 (B051J81, 5′-TCATCAAAGGAAGGCACTC C-3′ and 051STOP-R, 5′-TAGATATGAAGGAGGTCAGAG-3′), cyclinB (CYM F, 5′-AGTGGTACTTAACAGTTCCAACACC-3′ and CYM R, 5′-AGAGAACCTCACCAAACATTGCCTG-3′). In situ hybridization was performed as described elsewhere (Semiarti et al. 2001), with the exception that plant material was fixed overnight at 4° C. in 4% paraformaldehyde and 0.25% glutaraldehyde in 0.1 M sodium phosphate buffer (pH 7.4) (Sakamoto et al. 2001). A 827-bp product of PCR of NACK1 cDNA was cloned into pBluescript (SK−) (Stratagene, La Jolla, Calif., USA) to generate pNU640. An anti-sense RNA probe was generated by linearizing pNU640 with SalI, with subsequent synthesis of RNA by T3 RNA polymerase.

For analyses of RNA levels in Arabidopsis by real-time PCR, we prepared polyadenylated RNA from 10 μg of total RNA. Reverse transcription was performed using Ready-To-G0 You-Prime First-Strand Beads (Amersham). We used primers specific for S™ (STM-1, 5′-CTCCTCCCCAAGGAACTAAGAAC-3′ and STM-2, 5′-TCCTCCTGCAACGATTTCG-3′), BP (BP-1, 5′-TGTTGTTTCCACATATGAGCTCTCT-3′ and BP-2, 5′-TCATGATCAGATCGGAAGCAAT-3′), KNAT2 (KNAT2-1, 5′-TTCCGCTCGACGGAAGAC-3′ and KNAT2-2, 5′-AATCGGACGGCATCATCAAC-3′), KNAT6 (KNAT6-1, 5′-GATGTCACCGGAGAGTCTCATG-3′ and KNAT6-2, 5′-CGGCGGAGGAACATAGCA-3′), CUC1 (CUC1-1, 5′-TTGCTCCGATCATCAATACCTTT-3′ and CUC1-2, 5′-CATCGGTATGAGCAGCAGAGTT-3′), CUC2 (CUC2-1, 5′-CACAGCCAGCGCAATAACC-3′ and CUC2-2, 5′-TCTAAGCCCAAGGCCGTAGTAG-3′), CUC3 (CUC3-1, 5′-CGAACTCGCCGGAGAAGA-3′ and CUC3-2, 5′-TCGTCCGTCGGGTGAAAC-3′), AtNACK1 (NACK)-1, 5′-CCAAGCAGCGCATCCAA-3′ and NACK1-2, 5′-AAGACTTGCCTAGAAGCTGAAAGC-3′) and CYCB (CYCB-1, 5′-GAAAGATGGTTGGTTTGCATCA-3′ and CYCB-2, 5′-TGGATGTGTTGTATTTCCTGTGAA-3′).

PCR was carried out in the presence of the double-strand DNA-specific dye SYBR green (Applied Biosystems). Amplification was monitored in real time with the 7500 Real Time PCR System (Applied Biosystems).

### Microscopy

For anatomical analyses of tobacco leaves, plants were grown for 45 d as described above, leaves with lengths of 5-6 cm were isolated from the wild-type and AK-6b transgenic plants and used for preparing plastic sections as described elsewhere (Tanaka et al. 2001). Subcellular localization of sGFP fusion protein was observed as described elsewhere (Nishihama et al. 2001).

Followings are applied to examples 4 to 6.

### Transformation.

Tobacco leaf discs and BY-2 cells were transformed with Agrohacterium tumefaciens LBA4404 by previously described procedures8, 28 and above plant material.

### Yeast Two-Hybrid Screening.

Screening was performed basically as described previously8. A cDNA library was synthesized from poly(A)+ RNA that had been isolated from 3-week-old tobacco plants by the standard protocol with a TimeSaver™ cDNA synthesis kit (Amersham Biosciences, Buckinghamshire, UK).

### Construction of Plasmids.

Plasmid DNAs were constructed by amplification by PCR and standard cloning techniques. Details of procedures are available upon request.

### Isolation of Nuclei.

Nuclei were isolated from BY-2 cells as described previously29. BY-2 cells were washed twice with buffer A [(9% (w/v) sorbitol, 25 mM Mes (pH 5.6), 5 mM CaCl2) and suspended in an enzyme mixture [9% (w/v) sorbitol, 3% (w/v) cellulase Onozuka R-10 (Yakult, Tokyo, Japan), 1% (w/v) Macerozyme R10 (Yakult), pH 5.5], and incubated at 25° C. for 40 min. After cells had been washed with buffer A, they were suspended in ice-cold buffer B [25 mM Mes (pH 5.6), 5 mM MgCl2, 10 mM KCl, 0.35 M sucrose, 30% (w/w) glycerol, and a protease inhibitor cocktail (Roche, Mannheim, Germany)] and then homogenized with a glass homogenizer. The homogenate was filtered successively through a series of stainless-steel screens of decreasing pore diameter (37 micro m, 25 micro m, and 22 micro m) and the screens were washed with buffer C [buffer B containing 4% (w/v) Triton X-100]. The nuclei in the final filtrate were collected by centrifugation and the pellet was resuspended in buffer C. The nuclei were subsequently purified twice by Percoll density gradient centrifugation. The final preparation of nuclei was suspended in a buffer that contained 50% glycerol, 1 mM Dithiothreitol (DTT), 10 mM KCl, 10 mM MgCl2 and 20 mM Mes (pH 6.0) and stored at −80° C. until use. All manipulations were performed at 4° C.

### Analysis of Protein-Protein Interactions.

The interaction between histone H3 and 6b was examined essentially as described elsewhere8, with the exception that the binding buffer was replaced by TBS that contained 10 mM MgCl2 and a protease inhibitor cocktail. Isolated nuclei (1×10⁶) were resuspended in 500 micro 1 of MNase buffer [20 mM Tris-HCl (pH 7.5), 100 mM KCl, 2 mM MgCl2, 1 mM CaCl2, 0.3M sucrose, 0.1% Triton X-100, 3 units/micro 1 micrococcal nuclease (MNase; Amersham Biosciences, Buckinghamshire, UK) (MNase), and a protease inhibitor cocktail], and incubated at 25° C. for 180 minutes. The reaction was stopped by addition of ethylenediaminetetraacetate (EDTA) and ethyl ethylene glycol-bis(beta-aminoethyl ether)-N,N,N′,N′-tetraacetate (EGTA) to give a final concentration of 5 mM each. Protein complexes were immunoprecipitated with T7-specific antibody-conjugated agarose beads and the agarose beads were then washed three times with TBS. Bound proteins were eluted by boiling in loading buffer for SDS-PAGE and then subjected to SDS-PAGE and Western blotting analysis.

### Isolation of Chromatin.

The method for the isolation of chromatin has been described previously28. Isolated nuclei were disrupted in lysis buffer [10 mM PIPES-HCl (pH 6.8), 10 mM EDTA, and the protease inhibitor cocktail], and centrifuged at 6,000×g for 10 min. The pellet and the supernatant were recovered as the chromatin fraction and the nucleoplasmic fraction, respectively.

### Salt Extraction.

Nuclei (1×10⁶) were incubated in 100 micro 1 of lysis buffer that contained NaCl at various concentrations. After a 30-min incubation on ice, samples were centrifuged at 12,800×g for 15 min at 4° C. to yield the supernatant (S) and pellet (P) fractions.

### Supercoiling Assay.

Recombinant His-T7-6b and hNAP-1 protein were produced in E. coli cells, affinity-purified on HIS-Select cobalt affinity gel (Sigma, St. Louis, Mo., USA) and glutathione Sepharose (Amersham Bioscience) and then subjected to chromatography on Q Sepharose Fast-Flow (Amersham Bioscience). Core histones were purified from HeLa cells as described previously30. Relaxed pBR322 plasmid DNA (0.2 micro g), core histones and recombinant hNAP-1 and His-T7-6b were incubated in 30 micro 1 of assembly buffer [10 mM Tris-HCl (pH 7.5), 100 mM NaCl, 2 mM MgCl2, 0.1% bovine serum albumin, and 2 units of topoisomerase I (Invitrogen, Carlsbad, Calif., USA)] for 45 min at 37° C. The reaction was stopped by addition of 3 micro 1 of stop buffer (2% SDS and 0.5 mg/ml proteinase K) and incubation at 37° C. for 15 min. After the reaction, plasmid DNA was extracted with phenol and chloroform and precipitated in ethanol. Plasmid DNA was subjected to agarose gel electrophoresis (1% agarose), and visualized by staining with ethidium bromide.

### Example 1

### AK-6b Gene Stimulates Cell Division and Affects Cell Differentiation at the Abaxial Side of Leaves

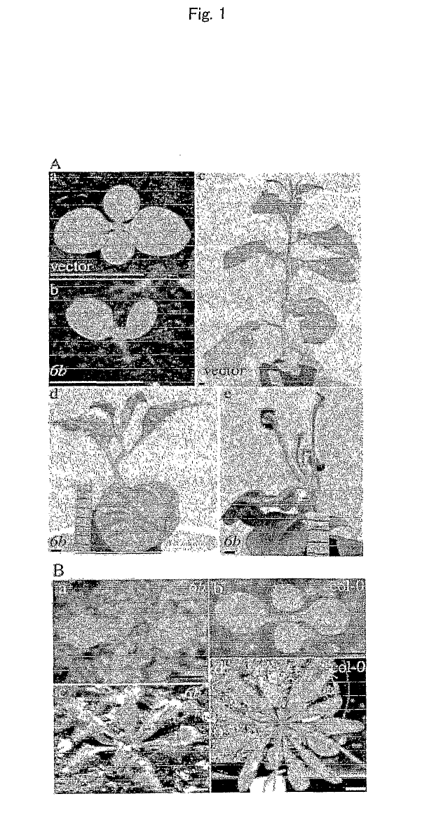

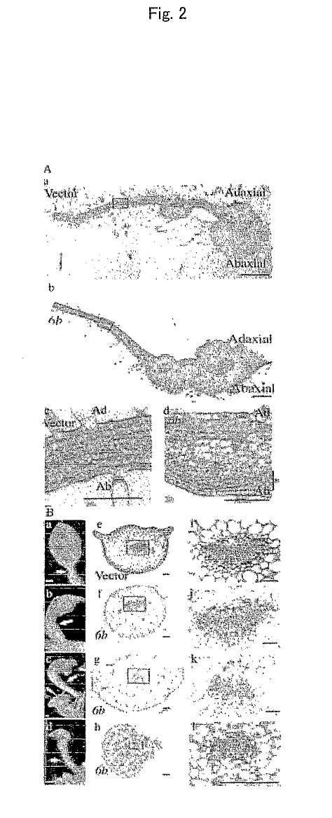

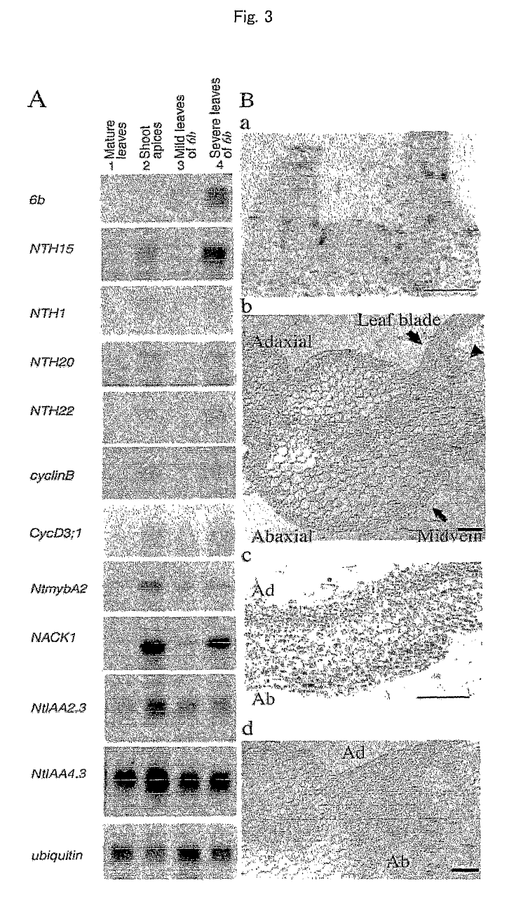

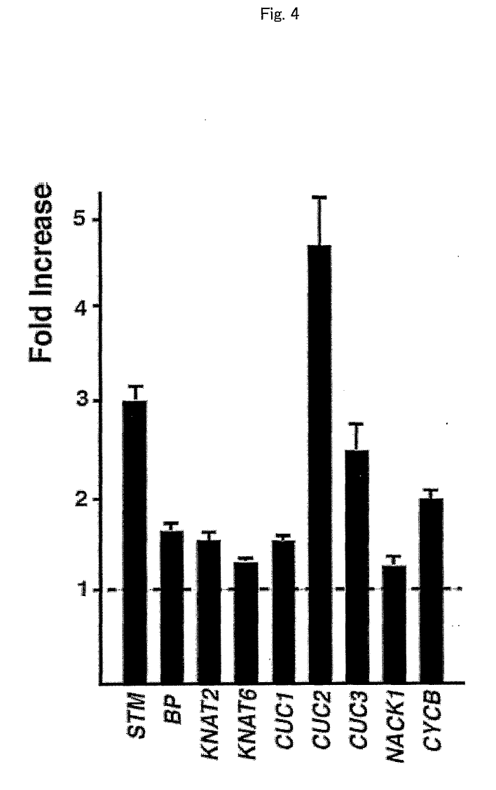

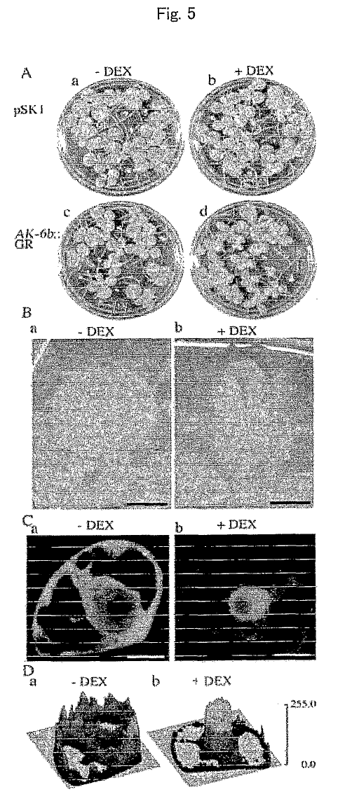

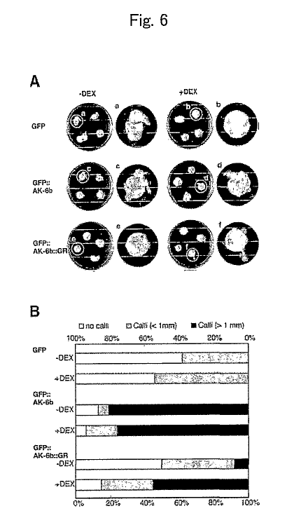

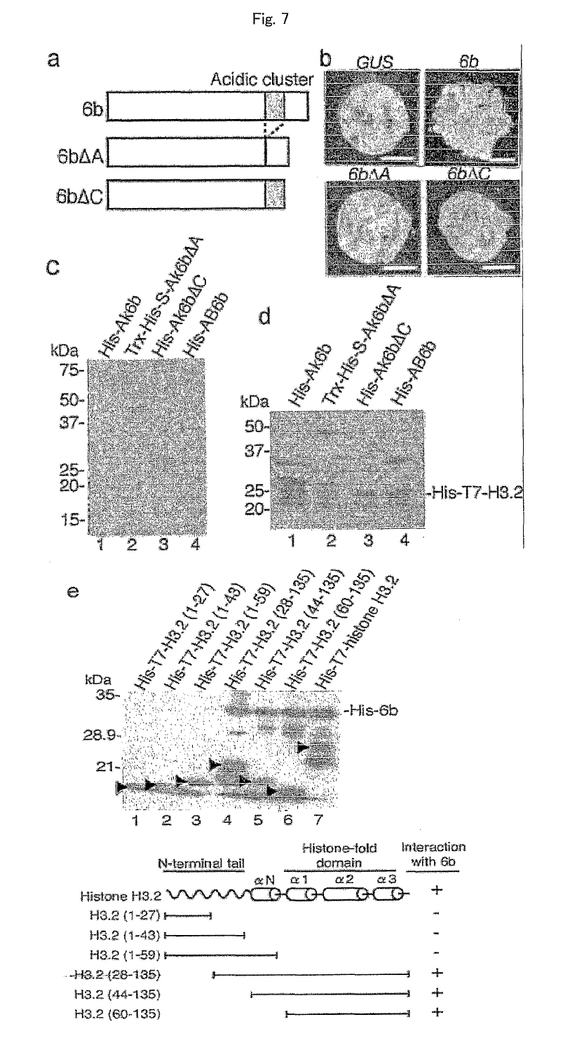

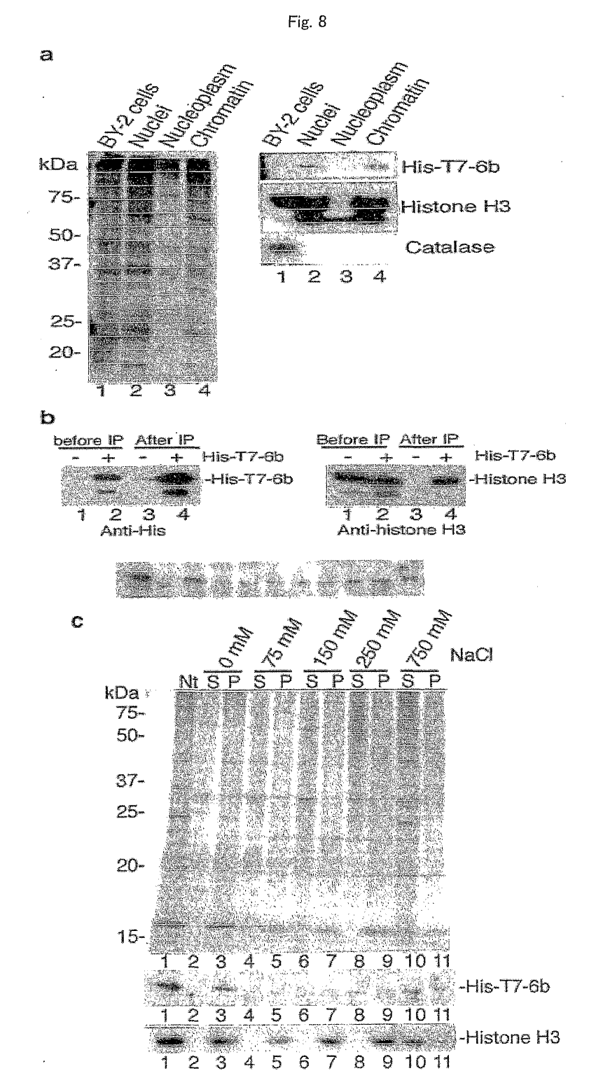

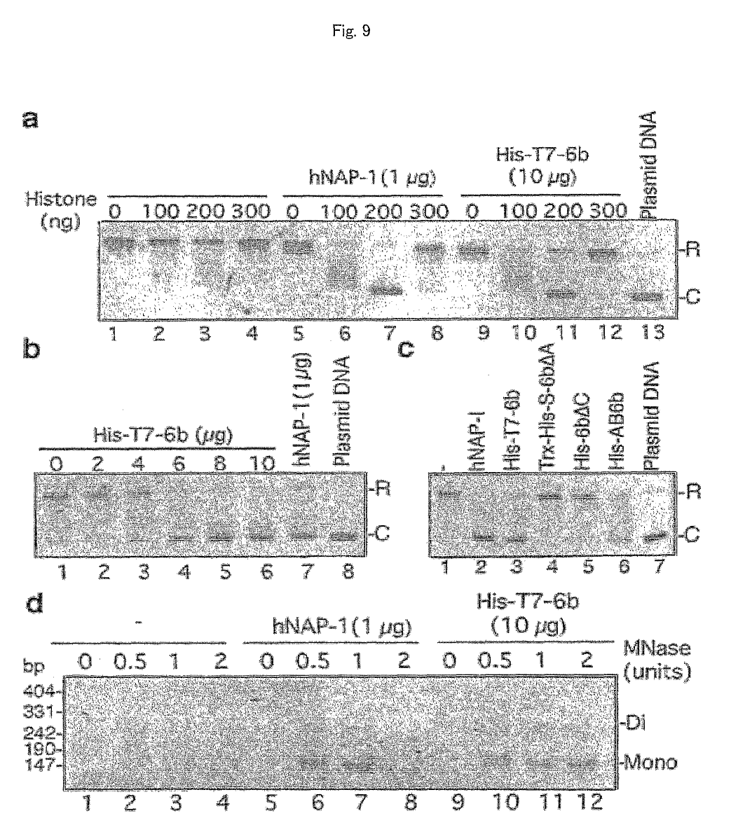

Transgenic tobacco plants that express the AK-6b gene controlled by the 355 promoter (P35S) exhibited two classes of phenotypes. In tobacco plants that displayed a mild phenotype, cotyledons and leaves were curled upwardly along the longitudinal axis at early growth stages (FIGS. 1Ab and Ad), and generated a number of outgrowths from the abaxial surface (FIG. 1Ad). Plants that showed a severe phenotype produced leaves with long petioles and an unexpanded lamina that was often associated with rod-shaped protrusions (FIG. 1Ae). Transgenic Arabidopsis plants generated upwardly curled cotyledons and rosette leaves, which often 7 exhibited extensive serration (FIG. 1B). Thus, upward curling of cotyledons and leaves along the longitudinal axis was the common phenotype in transgenic tobacco and Arabidopsis.

Because of obvious leaf abnormality of transgenic tobacco, we carried out anatomical studies with such transgenic plants. We prepared transverse sections of the upwardly curled leaves (FIG. 1Bb) and investigated numbers and shapes of mesophyl1 cells (FIG. 2). Compared with sections from wild-type leaves (FIGS. 2Aa, 2Ac), the number of cell layers in transgenic leaves was approximately 1.5-fold more in the adaxial/abaxial orientation: roughly three layers were added (FIGS. 2Ab, 2Ad). This seems to be due to the presence of additional layers of small cells at the abaxial side (FIG. 2Ad).

We also observed cross sections of the petiole in AK-6b transgenic tobacco plants (FIG. 2B). In the cross section from the middle region of a petiole of AK-6b-expressing tobacco that exhibited the mild phenotype (FIG. 1Ad), many clusters of densely stained cells were visible in cortex cells (FIGS. 2Bf and 2Bj). In the petiole of severely affected plants (FIG. 1Ae), organizations of epidermis, cortex and vascular tissues were distorted, a prominent mid vein disappeared and a number of clusters of small and densely stained cells appeared in cortex (FIGS. 2Bc, 2Bg, 2Bd and 2Bh).

In such a severely distorted and long petiole, the establishment of the adaxial/abaxial polarity seemed to be disrupted, because of the lack of leaf blade and the disappearance of the normally arrayed vascular bundles along the adaxial/abaxial axis (FIG. 2B1).

### Example 2

### Cell Division and Meristem-Related Genes are Ectopically Expressed in AK-6b Transgenic Tobacco and Arabidopsis Plants

Because phenotypic abnormalities in leaves were related to cell division and differentiation as we described above, we prepared RNAs from leaves with 5-6 cm lengths that were isolated from wild-type and 6b transgenic tobacco plants grown as described in Materials and Methods and investigated transcription of genes involved in cell division and development and maintenance of the shoot apical meristem. As shown in FIG. 3A, levels of AK-6b transcripts in leaves of transgenic tobacco were correlated with severity of the phenotype (lanes 3 and 4).

We examined levels of transcripts of NTH15, NTH1, NTH20 and NTH22 genes, members of the class 1 KNOX homeobox gene family of tobacco (Nishimura et al. 1999). Transcripts of these homeobox genes accumulated in leaves of the severe phenotype (FIG. 3A, lane 4) as well as in shoot apices of untransformed tobacco (FIG. 3A, lane 2), but transcripts were rarely detected in leaves showing the mild phenotype and in untransformed leaves (FIG. 3A, lanes 3 and 1). Transcripts of cyclinB, CycD3; 1, NtmybA2 and NACK1 genes that normally express only at certain stages of the cell cycle also accumulated in transgenic leaves (lanes 3 and 4) and shoot apices of untransformed tobacco (lane 2).

The accumulation levels of transcripts of IAA2.3 and IAA4.3 genes in transgenic leaves did not differ from those of transcripts of the corresponding genes in untransformed leaves.

We performed in situ hybridization to detect transcripts of NACK1 that encode an M-phase-specific kinesin-like protein in leaves as isolated above. In untransformed tobacco, NACK1 transcripts accumulated in a patchy pattern in the region around a shoot apical meristem and leaf primordia (FIG. 3Ba), which was similar to the pattern of cyclinB and NtmybA2 transcripts (Ito et al. 2001). In AK-6b transgenic tobacco, positive signals were detected in the junction region between the midvein and the leaf blade of the abaxial side (FIG. 3Bb) and in the additional cell layer of the abaxial side of the leaf blade (FIG. 3Bc). No such signal was detected in the adaxial side of AK-6b transgenic leaves.

We also investigated by real-time polymerase-chain reaction (PCR) accumulation levels of transcripts of Arabidopsis genes such as class 1 KNOX genes (STM, BP, KNAT2, KNAT6), CUC1, CUC2, CUC3, AtNACK1/HIK and cyclinB genes in the first and the second leaves of 14 days old wild-type and AK-6b-transgenic Arabidopsis plants. As shown in FIG. 4, levels of transcripts of these genes increased in the leaves of the AK-6b transgenic Arabidopsis although rates of increase were different in the genes examined.

### Example 3

### The Nuclear Import of AK-6b Protein is Crucial for Upward Curling of Transgenic Leaves and In Vitro Formation of Calli

To examine a correlation between nuclear localization of the 6b protein and the phenotypes created by expression of 6b in tobacco plants, we utilized a fusion protein between AK-6b and the glucocorticoid receptor (GR), nuclear import of which can be induced by the steroid hormone dexamethasone (DEX). Morphology of transgenic tobacco plants that expressed the AK-6b::GR fusion gene was indistinguishable from that of control tobacco plants transformed with empty vector PSK1 in the absence of DEX (FIGS. 5Aa, 5Ac and 5Ba). When transgenic plants with AK-6b::GR were grown in the presence of DEX, they generated upwardly curled leaves (FIGS. 5Ad and 5Bb). Transgenic tobacco plants transformed with an empty vector did not show such a phenotype even in the presence of DEX (FIG. 5Ab). We obtained 12 independent transgenic tobacco plants and 6 of them showed the DEX-dependent phenotype.

These results suggest that the AK-6b-GR protein that may exist in the cytoplasm without DEX is not functional in the generation of phenotypes and that its nuclear import induced by DEX causes the appearance of the phenotype. Transgenic tobacco plants that express GVG-GR exhibit no morphological abnormality (Nishihama et al., 2001), indicating that the GR moiety in the fusion protein was not responsible for the phenotype observed.

To examine whether the functional conversion of AK-6b activity induced by DEX is correlated with the nuclear localization of AK-6b-GR, we generated a fusion construct in which the AK-6b::GR DNA was fused to the gene for green fluorescent protein (sGFP) (sGFP::AK-6b::GR). The fusion construct was introduced into BY-2 cells and fluorescence due to sGFP in the cells was monitored. Although the signal was distributed in cytoplasm in the absence of DEX (FIG. 5Ca), the signal of the sGFP-AK-6b-GR fusion protein was detected in nuclei of BY-2 in the presence of DEX (FIG. 5Cb). Quantitative analysis of the fluorescence signals showed the clear nuclear localization of the fusion protein that was induced by DEX (FIG. 5D).

Subsequently, we examined effects of DEX on callus formation from transgenic leaves carrying sGFP-AK-6b-GR. When the leaf discs carrying sGFP-AK-6b were incubated in hormone-free medium, calli were formed regardless of the presence of DEX (FIGS. 6Ac, 6Ad and B). However, such calli were generated only in the presence of DEX when discs of transgenic leaves carrying FP-AK-6b-GR were tested (FIGS. 6Ae, 6Af, and B). These results suggest that the nuclear import of AK-6b protein is important for the generation of upwardly curled leaves in AK-6b transgenic plants and hormone-independent formation of calli.

### Discussion

### Overexpression of the AK-6b Gene Induces Ectopic Cell Division in Leaves of Transgenic Tobacco

The present results provided three lines of evidence for occurrence of ectopic cell division and the altered meristematic state of cells at least in the abaxial side of leaves and in petioles of AK-6b transgenic tobacco plants. First, microscopic analysis of transgenic leaves showed that the abaxial side of the leaves contained a large number of additional small cells as well as enation-like protrusions in the abaxial side (FIG. 2). These phenotypic observations are consistent with those reported by Helfer et al. (2003). Petioles of transgenic tobacco plants also contained a number of smaller cells, compared with cells in untransformed tobacco plants, and many clusters of densely stained cells, which are similar to cells of vascular tissues (FIG. 2). Second, transcripts of meristem-related homeobox genes and cell division-controlling genes, which are normally accumulated in dividing cells and meristematic tissues but not in mature leaves, were detected in leaves of transgenic tobacco and the Arabidopsis plants (FIGS. 3 and 4). Levels of transcripts of these genes were well correlated with severity of phenotypes generated by AK-6b expression (FIG. 3A). Helfer et al. (2003) reported that the transcript levels of the cell cycle genes such as NtCYC1 (cyclin B1) in leaves of AB-6b-expressing tobacco are rather lower than those in leaves of normal tobacco when leaves in the same developmental stages are examined. Observed differences in transcript levels might have been due to lower accumulation levels of AB-6b transcripts than those of AK-6b transcripts in the present study. Third, analysis by in situ hybridization revealed that transcripts of the M-phase-specific gene NACK1 accumulated in a patchy pattern in the abaxial side of transgenic leaves (FIG. 3B). These results indicate that cell proliferation is stimulated and the developmentally indeterminate state increases in leaves of transgenic plants expressing the AK-6b gene, particularly in the abaxial side. Such an unbalanced cell proliferation between adaxial and abaxial sides of leaves might cause upward curling of the leaves, which was observed commonly both in transgenic tobacco and Arabidopsis plants (FIG. 1). Similar upward curling of leaves is also observed in Arabidopsis plants that overexpress the ASYMMETRIC LEAVES2 (AS2) gene (Iwakawa et al. 2002), co-express the ipt, iaaM and iaaH genes from the T-DNA (see Introduction) (Eklöf et al. 2000), and have gain-of-function mutations in some IAA genes that are negative regulators of auxin-inducible transcription of genes (Reed 2001). Therefore, one of the roles of 6b might be related to physiological processes controlled by these genes.

Recently, we have identified several genes for tobacco nuclear proteins that bind the AK-6b protein and proposed that these plant proteins might be involved in expression of plant genes that might be related to cell proliferation (Kitakura et al. 2002). Such AK-6b-interacting factors in plant cells might contribute to the tissue specificity of the cell proliferation generated by AK-6b, although tissue-specific roles and/or localization of these factors have yet to be determined. The molecular mechanism behind the unbalanced growth of cells remains to be elucidated.

Although the 6b gene has the ability to induce the proliferation of plant cells (see Introduction), the effect of 6b on the formation of crown galls is relatively weak as compared to those of genes for biosynthesis of auxin [iaaM (tms1) and iaaH (tms2)] and cytokinin [ipt (tmr)] in T-DNAs: mutations in 6b do not severely affect the tumorigenicity by T-DNAs (Garfinkel et al. 1981; Joos et al. 1983). However, it is worth noting that the 6b gene is widely conserved in T-DNA regions of various Ti plasmids (Helfer and Otten 2002). Such conservation suggests a certain role of 6b in the tumor formation that is associated with the genetic transformation with T-DNAs. It was proposed that the gene might have a more crucial role in tumorigenicity on some host plant species (Hooykaas et al. 1988). Molecular analysis of the 6b protein in nuclei will provide further understandings of a role of this protein in the tumor formation on plants.

### The AK-6b Gene Inhibits Development of Leaf Blades and Proper Vascular Systems

The high extent of AK-6b expression in the severe mutant leaves inhibited development of leaf blades and generated rod-like leaf structures with narrow and flat regions at the tips (FIG. 1Ae). In addition, a single prominent vascular system that is normally present in the middle of a petiole was not found, and many thinner vasculatures were observed in the rod-like leaves (FIGS. 2Bd and 2Bh). These observations may imply that the formation of leaf blades and the proper development of vasculatures are severely inhibited by the expression of AK-6b. Since these phenotypes are reminiscent of malformed leaves generated by overexpression and ectopic expression of YANADI genes of Arabidopsis that determine the abaxial fate of leaves (Eshed et al. 2001), and by multiple loss-of-function mutations in PHABULOSA, PHAVOLUTA and REVOLUTA genes that are determinants for the adaxial fate of leaves (Emery et al. 2003), the adaxial/abaxial polarity of these transgenic tobacco leaves seems to be affected by expression of the AK-6b gene. It is proposed that leaf blades would be generated on the basis of the development of the adaxial-abaxial polarity of leaves: if the development of such a polarity is disordered by loss-of-function mutations in genes responsible for polarity formation, formation of leaf blades is inhibited, which results in generation of rod-like leaves with malformed vascular tissues (Waites and Hudson 1995; Emery et al. 2003). However, levels of transcripts of these genes were not significantly affected in AK-6b transgenic Arabidopsis (our unpublished data). The observed alteration generated by AK-6b seems to be independent of the control by the mechanism modulated by these genes. The relationship between the 6b gene and the adaxial/abaxial polarity must be further studied.

Roles of class 1 KNOX homeobox genes in formation of abnormal leaves by AK-6b number of class 1 KNOX homeobox genes were ectopically accumulated in leaves of transgenic tobacco and Arabidopsis (FIGS. 3 and 4). SHOOT-MERISTEMLESS gene (STM) is known to be responsible for development and/or maintenance of shoot apical meristems during plant development. Other related genes are also normally expressed in or around the shoot apical meristem (Tamaoki et al. 1997; Nishimura et al. 1999; Hake et al. 2004; Long et al. 1996).

Transcripts of class 1 KNOX genes were hardly detected in mature leaves. Overexpression and ectopic expression of these genes cause formation of malformed leaves with knobs, lobes and serrations, and also induce formation of ectopic shoots, suggesting that KNOX genes induce the conversion of differentiated states of cells to meristematic states in leaves (Tamaoki et al. 1997; Nishimura et al. 2000; Vollbrecht et al. 1991; Byrne et al. 2000; Semiarti et al. 2001). Some phenotypes such as the enation-like protrusion and the abnormality of the adaxial/abaxial polarity of leaves generated by AK-6b might be related to ectopic expression of these KNOX genes in tobacco leaves. Expression of the orf13 gene, which is present on the T-DNA of Agrobacterium rhizogenes and encodes a protein that is similar in terms of amino acid sequence to 6b, also induces ectopic expression of class 1 KNOX genes in mature leaves of tomato (Stieger et al. 2004). They propose that orf13 could confer meristematic competence to cells infected by A. rhizogenes by inducing the expression of KNOX genes. To understand the molecular basis of the leaf abnormalities, the roles of class 1 KNOX genes need further investigation. In addition, it would be intriguing to study the mechanism by which the AK-6b protein can induce expression of these homeobox genes, although it is uncertain whether AK-6b may control the expression directly or indirectly. The observation that nuclear import of AK-6b protein is required for the appearance of phenotypes (FIGS. 5 and 6) suggests a role of this protein in nuclei. We have previously shown that a fusion protein composed of the DNA-binding domain of yeast GAL4 and AK-6b activates transcription of a reporter gene in tobacco cells (Kitakura et al. 2002). Therefore, the 6b protein has a potential to directly affect expression of certain genes that might be involved in cell proliferation and differentiation. Investigation of molecular mechanisms of the processes stimulated by 6b protein should provide a new insight for understanding normal cellular systems that control cell growth and differentiation in plants.

### Example 4

The 6b protein includes a cluster of acidic amino-acid residues near its carboxyl terminus (FIG. 7a). This acidic region is required both for the interaction of 6b with the tobacco nuclear protein NtSIP1 and for the induction by 6b of the formation of calli on hormone-free medium8 (FIG. 7b). To identify tobacco proteins that might bind to regions of 6b other than the acidic region, we screened a tobacco cDNA library (1.5×10⁶ independent clones) in a yeast two-hybrid system, using the 6b delta A sequence (FIG. 7a) that lacks the acidic region. We identified five positive clones. Two of the cDNA clones encoded members of the histone H3 family (histone H3.1 and histone H3.2)16; one clone encoded an amino acid sequence that was similar to the amino acid sequence of the histone-fold domain of histone H2B; and two clones encoded proteins with no significant homology to previously characterized sequences.

We performed coimmunoprecipitation experiments with His epitope-tagged 6b (His-6b) and His epitope- and T7 epitope-tagged tobacco histone H3.2 (His-T7-histone H3) to examine their interactions in vitro. In these binding assays, we used two kinds of 6b protein: one from A. tumefaciens AKE10 (designated AK-6b) and one from A. vitis AB4 (designated AB-6b) because these 6b proteins, from two different bacterial sources, are both able to induce abnormal morphology in tobacco and Arabidopsis plants-6,9,17.

We also generated Trx-His-S-AK-6b delta A and His-AK-6b delta C proteins, which lacked the carboxy-terminal region of 6b proteins (FIG. 7a) and were unable to support planthormone-independent growth (FIG. 1b). We found that AK-6b, AK-6b delta A, and AB-6b bound histone H3.2, while AK-6b delta C did not (FIG. 7b). These results indicated that the carboxy-terminal region of AK-6b was required for the binding to histone H3. Arabidopsis histone H3.1 and other types of human histone (H2A, H2B, and H4) also bound to both wild-type 6b proteins in vitro (data not shown).

We attempted to identify the regions in histone H3.2 that might be required for the interaction of this histone with 6b in vitro. We prepared various truncated derivatives of histone H3.2 from Eseherichia coli cells that produced these proteins, and the structures of these proteins are shown schematically in FIG. 1e. Our results demonstrated that AK-6b protein bound to histone H3.2 (28-135), histone H3.2 (44-135), and histone H3.2 (60-135), all of which contain the so-called histone-fold domain, which appears to be involved in the histone/histone and histone/DNA interactions that are involved in the formation of nucleosome structures, but not the region that contained the histone tail and the amino-terminal alpha helix.

### Example 5

To examine the interaction of AK-6b with histone H3 in vivo, we isolated nuclei from cells of the tobacco cultured cell line BY-2 that produced His-T7-6b and then we separated the chromatin fraction from the nucleoplasmic fraction. We performed Western blotting analysis of the nuclear, chromatin and nucleoplasmic fractions with histone H3-specific and T7-specific antibodies (FIG. 8a). We used catalase-specific antibodies to detect catalase as a marker for cytoplasmic proteins. Our results showed that the nuclear and chromatin fractions contained both histone H3 and His-T7-6b proteins but the nucleoplasmic fraction did not (FIG. 8a), indicating that AK-6b can associate with tobacco chromatins.

We also performed an immunoprecipitation assay by adding T7-specific antibodies to the chromatin fraction. Western blotting with histone H3-specific antibodies showed that the immunocomplex contained the endogenous histone H3 of tobacco (FIG. 8b), revealing a physical interaction between AK-6b and histone H3 in tobacco cells.

We compared the affinity of His-T7-6b for chromatin to that of histone H3 by treating chromatin fractions with various concentrations of NaCl and examining the amounts of these proteins in NaCl-soluble (supernatant; S) and NaCl-insoluble (pellet; P) fractions. Western blotting analysis showed that, as the concentration of NaCl increased, the amount of His-T7-6b and the amount of histone H3 released from the chromatin gradually increased (FIG. 8c). We noted, however, a difference in the amount of each protein released from the chromatin: endogenous histone H3 was not released to any significant extent from the chromatin at 75, 150 and 250 mM NaCl even though some of the His-T7-6b protein was clearly released from the chromatin at these concentrations of NaCl. These results suggested that these proteins bound with somewhat different affinities to the chromatin.

The physical association of 6b with the histone fold of histone H3 and the presence of the acidic region in 6b led us to examine 6b for histone-chaperone activity since some histone chaperones, such as HIRA and ASF1, bind to the histone-fold domains of core histones18,19 and, furthermore, histone chaperones, such as NAP-120, nucleophosmin/B2321, and yeast FK506 bp22, contain clusters of acidic amino-acid residues. Histone chaperones enhance the formation of nucleosomes in vitro, this activity can be detected by DNA supercoiling assays, which measure the conversion of relaxed and closed circular DNA to negatively supercoiled DNA, and treatment with microccocal nuclease23,24.

### Example 6

To examine the putative histone-chaperone activity of 6b protein, we first performed supercoiling assays with relaxed and closed circular DNA, AK-6b or AB-6b proteins that had been purified from E. coli cells that produced these proteins, and core histones prepared from HeLa cells.

FIGS. 9a and 9c show that both AK6b and AB6b had supercoiling activity, with the maximum activity detected when we used 10 micro g of purified AK-6b or AB-6b and 200 ng of core histones. When we used 1 micro g of human NAP-1 (hNAP-1) protein, which has been shown to have histone-chaperone activity, maximum activity was also detected with 200 ng of core histones. Since the molar ratio of core histones to plasmid DNA under our conditions was 1:1, which corresponds to the normal ratio in the supercoiling reaction, we can reasonably assume that the activity that we detected might be relevant to normal reactions mediated by a histone chaperone25. However, we had to add approximately fifteen times more His-T7-6b than hNAP-1 for maximum conversion to negative supercoils (FIG. 9b). As shown in FIG. 9c, the mutant derivatives of 6b that lacked either the acidic region (6b delta A) or the carboxy-terminal region (6b delta C) had no histone-chaperone activity.

To examine the possible formation of nucleosome structures, we digested the plasmid DNA, after the supercoiling reaction, with microccocal nuclease (MNase). As shown in FIG. 9d, DNA fragments of distinct lengths (approximately 140 bp and 280 bp) were generated, indicating that nucleosome structures had been formed in the presence of 6b or hNAP-1 and core histones. Our observations indicated that 6b had histone-chaperone activity in vitro but that the activity of 6b was lower than that of hNAP-1 in our assay system.

### Discussion

A number of proteins that have histone-chaperone activity can mediate the assembly and disassembly of nucleosomes. Such proteins might affect the structure of the chromatin, and alterations in the structure of the chromatin might, in turn, influence nuclear events, such as transcription and DNA replication26. The 6b protein might similarly affect the structure of chromatin, inducing alterations in patterns of gene expression in 6b-transformed plants, which might normally be regulated by developmental programs intrinsic to the host plants and/or by the actions of phytohormones, such as auxin and cytokinin. The reported observation that genes involved in the proliferation of plant cells and the differentiation of plant organs are expressed ectopically in 6b-transgenic plants9 is consistent with this hypothesis. It is important, now, to identify genes whose transcription is directly affected by 6b if we are more fully to understand the functions of this protein.

The present results should provide some new insight into the roles of 6b in the formation of crown gall tumours. We propose the following possible roles for 6b. (1) The 6b protein might influence the structure of chromatin around the hormone synthesizing genes in T-DNA to allow the efficient expression of these genes, which might stimulate the formation of crown gall tumours. However, this scenario is somewhat unlikely because mutations in 6b have only a slight effect on the size and morphology of crown galls generated in the laboratories but not essentials. A more plausible role for 6b is in guaranteeing the stable expression of the genes in T-DNA for long-term maintenance of crown galls on plants. Plants exploit a variety of gene silencing systems that are based on alterations of chromatin structures. These systems involve various histone modifications, DNA methylation, and small RNAs, such as miRNAs and siRNAs27. Such silencing of the phytohormone-synthesizing genes in the T-DNA in plant genomes might have a negative effect on the maintenance of crown galls. The 6b protein might play a role in maintenance of the chromatin in a state that favours the long-term expression of T-DNA genes and in protecting such expression from the silencing systems in plant cells. (2) Alternatively, 6b might play a role in modulating the expression of phytohormone-regulating genes through alterations in of chromatin structure such that the gene expression induced by phytohormones favours to the formation and maintenance of crown galls. We do not know how 6b might modulate the phytohormone-inducible expression of genes in plant cells. However, it is clear that modulation of chromatin structure must be crucial for the expression of plant genes that are regulated by auxin and cytokinin.

### Example 7

To examine the expression pattern when 6b protein introduced into plant, we examined the expression level by Array system and Real-time PCR system. These results are shown in Table 1 and Table 2.

The wild type Arabidopsis seeds and the Arabidopsis seeds expressing a wild type and 6b were subject to sterilization treatment (1% hyoichlorous acid, 0.02% Triton X-100) at room temperature for 5 minutes using sterilization liquid, were washed 3 times with sterilization liquid, and were seeded in the Murashige/Skoog medium of 0.2% grangum. The seeds were subject to low-temperature treatment for days with 4□, were sprouted with 22□ in the bright light (6000 lux) for 16 hours and in the dark place for 8 hours, and were kept in warm atmosphere for 13 days. The plant bodies were picked up by forceps, and the aerial parts of the plant bodies including hypocotyl were collected by knife. Then, RNA was refined from the collected aerial parts (5 individuals) by using Rneasy Plant Mini Kit (QIAGEN, Hilden, Germany). Microarray analysis was performed in accordance with the protocol of The Agilent by using the refined RNA.

These expression profiles in plant expressing 6b gene disclosed that 6b protein modifies r alters the expression of IAA gene family members and TCP gene family members globally.

The contents of the following references are incorporated by reference herein in their entirety.
Byrne, M. E., Barley, R., Curtis, M., Arroyo, J. M., Dunham, M., Hudson, A. and Martienssen, R. A. (2000) Asymmetric leaves1 mediates leaf patterning and stem cell function in Arabidopsis. Nature 408: 967-971.
Dehesh, K., Hung, H., Tepperman, J. M. and Quail, P. H. (1992) GT-2: a transcription factor with twin autonomous DNA-binding domains of closely related but different target sequence specificity. EMBO J. 11: 4131-4144.
Eklöf, S., Astot, C., Sitbon, F., Moritz, T., Olsson, O. and Sandberg, G. (2000) Transgenic tobacco plants co-expressing Agrobacterium iaa and ipt genes have wild-type hormone levels but display both auxin- and cytokinin-overproducing phenotypes. Plant J. 23:279-84.
Emery, J. F., Floyd, S. K., Alvarez, J., Eshed, Y., Hawker, N. P., Izhaki, A., Baum, S. F. and Bowman, J. L. (2003) Radial patterning of Arabidopsis shoots by class III HD-ZIP and KANADI genes. Curr. Biol. 13: 1768-1774.
Eshed, Y., Baum, S. F., Perea, J. V. and Bowman, J. L. (2001) Establishment of polarity in lateral organs of plants. Curr. Biol. 11: 1251-1260.
Gális I, Kakiuchi Y, Simek P. and Wabiko H. (2004) Agrobacterium tumefaciens AK-6b gene modulates phenolic compound metabolism in tobacco. Phytochemistry 65: 169-79.
Garfinkel, D. J., Simpson, R. B., Ream, L. W., White, F. F., Gordon, M. P., and Nester, E. W. (1981) Genetic analysis of crown gall; fine structure map of the T-DNA by site directed mutagenesis. Cell 27: 143-153.
Grémillon, L., Helfer, A., Clément, B. and Otten, L. (2004) New plant growth-modifying properties of the Agrobacterium T-6b oncogene revealed by the use of a dexamethasone-inducible promoter. Plant J. 37: 218-228.
Hake, S., Smith, H. M., Holtan, H., Magnani, E., Mele, G. and Ramirez, J. (2004) The role of KNOX genes in plant development. Annu. Rev. Cell Dev. Biol. 20: 125-151.
Hamada, S., Onouchi, H., Tanaka, H., Kudo, M., Liu, Y. G., Shibata, D., Machida, C. and Machida, Y. (2000) Mutations in the WUSCHEL gene of Arabidopsis thaliana result in the development of shoots without juvenile leaves. Plant J. 24: 91-101.
Helfer, A. and Otten, L. (2002) Functional diversity and mutational analysis of Agrobacterium 6B oncoproteins. Mol. Gen. Genet. 267: 577-586
Helfer, A., Clement, B., Michler, P. and Otten, L. (2003) The Agrobacterium oncogene AB-6b causes a graft-transmissible enation syndrome in tobacco. Plant Mol. Biol. 52: 483-493.
Hooykaas, P. J. J., der Dulk-Ras, H. and Schilperoort, R. A. (1988) The Agrobacterium tumefaciens T-DNA gene 6b is an one gene. Plant Mol. Biol. 11: 791-794.
Ito, M., Araki, S., Matsunaga, S., Itoh, T., Nishihama, R., Machida, Y., Doonan, J. H. and Watanabe, A. (2001) G2/M-phase-specific transcription during the plant cell cycle is mediated by c-Myb-like transcription factors. Plant Cell 13: 1891-1905.
Iwakawa, H., Ueno, Y., Semiarti, E., Onouchi, H., Kojima, S., Tsukaya, H., Hasebe, M., Soma, T., Ikezaki, M., Machida, C. and Machida, Y. (2002) The ASYMMETRIC LEAVES2 gene of Arabidopsis thaliana, required for formation of a symmetric flat leaf lamina, encodes a member of a novel family of proteins characterized by cysteine repeats and a leucine zipper. Plant Cell Physiol. 43: 467-478
Joos, H., Caplan, A., Sormann, M., Van Montagu, M. and Schell, J. (1983) Genetic analysis of T-DNA transcripts in nopaline crown galls. Cell 32: 1057-1067
Kakiuchi, Y., Gális, I., Tamogami, S., Wabiko, H. (2005) Reduction of polar auxin transport in tobacco by the tumorigenic Agrobacterium tumefaciens AK-6b gene. Planta SEP 17: 1-11.
Kitakura, S., Fujita, T., Ueno, Y., Terakura, S., Wabiko, H. and Machida, Y. (2002) The protein encoded by oncogene 6b from Agrobacterium tumefaciens interacts with a nuclear protein of tobacco. Plant Cell 14: 451-463.
Kojima, S., Banno, H., Yoshioka, Y., Oka, A:, Machida, C. and Machida, Y. (1999) A binary vector plasmid for gene expression in plant cells that is stably maintained in Agrobacterium cells. DNA Res. 6: 407-410.
Long, J. A., Moan, E. I., Medford, J. I. and Barton, M. K. (1996) A member of the KNOTTED class of homeodomain proteins encoded by the STM gene of Arabidopsis. Nature 379: 66-69.
Nishihama, R., Soyano, T., Ishikawa, M., Araki, S., Tanaka, H., Asada, T., Irie, K., Ito, M., Terada, M., Banno, H., Yamazaki, Y. and Machida, Y. (2002) Expansion of the cell plate in plant cytokinesis requires a kinesin-like protein/MAPKKK complex Cell 109: 87-99.
Nishimura, A., Tamaoki, M., Sato, Y. and Matsuoka, M. (1999) The expression of tobacco knotted1-type class 1 homeobox genes corresponds to regions predicted by the cytohistological zonation model. Plant J. 18: 337-347.
Nishimura, A., Tamaoki, M., Sakamoto, T. and Matsuoka, M. (2000) Over-expression of tobacco knotted1-type class1 homeobox genes alters various leaf morphology. Plant Cell Physiol. 41: 583-590.
Reed, J. W. (2001) Roles and activities of Aux/IAA proteins in Arabidopsis. Trends in Plant Sci. 6: 420-425.
Sakamoto, T., Kamiya, N., Ueguchi-Tanaka, M., Iwahori, S, and Matuoka, M. (2001) KNOX homeodomain protein directly suppresses the expression of a gibberellin biosynthetic gene in the tobacco shoot apical meristem. Genes Dev. 15: 581-590.
Semiarti, E., Ueno, Y., Tsukaya, H., Iwakawa, H., Machida, C. and Machida, Y. (2001) The ASYMMETRIC LEAVES2 gene of Arabidopsis thaliana regulates formation of a symmetric lamina, establishment of venation and repression of meristem-related homeobox genes in leaves. Development 128: 1771-1783.
Tamaoki, M., Kusaba, S., Kano-Murakami, Y. and Matsuoka, M. (1997) Ectopic expression of a tobacco homeobox gene, NTH15, dramatically alters leaf morphology and hormone levels in transgenic tobacco. Plant Cell Physiol. 38: 917-927.
Tanaka, H., Onouchi, H., Kondo, M., Hara-Nishimura, I., Nishimura, M., Machida, C. and Machida, Y. (2001) A subtilisin-like serine protease is required for epidermal surface formation in Arabidopsis embryos and juvenile plants. Development 128: 4681-4689.
Tinland, B., Fournier, P., Heckel, T. and Otten, L. (1992) Expression of a chimeric heat-shock-inducible Agrobacterium 6b oncogene in Nicotiana rustica. Plant Mol. Biol. 18: 921-930.
Tinland, B., Huss, B., Paulus, F., Bonnard, G. and Otten, L. (1989) Agrobacterium tumefaciens 6b genes are strain-specific and affect the activity of auxin as well as cytokinin genes. Mol. Gen. Genet. 219: 217-224.
Vollbrecht, E., Veit, B., Sinha, N. and Hake, S. (1991) The de<d>velopmental gene Knotted-1 is a member of a maize homeobox gene family. Nature 350: 241-243.
Wabiko, H. and Minemura, M. (1996) Exogenous phytohormone-independent growth and regeneration of tobacco plants transgenic for the 6b gene of Agrobacterium tumefaciens AKE10. Plant Physiol. 112: 939-951.
Waites, R. and Hudson, A. (1995) phantastica: a gene required for dorsoventrality of leaves in Antirrhinum majus. Development 121: 2143-2154.

Following references cited with numbering in above description:
1. Garfinkel, D. J. et al., Genetic analysis of crown gall: fine structure map of the TDNA by site-directed mutagenesis. Cell 27, 143-153 (1981).
2. Willmitzer, L. et al. Size, location and polarity of T-DNA-encoded transcripts in nopaline crown gall tumors: common transcripts in octopine and nopaline tumors. Cell 32, 1045-1056 (1983).
3. Hooykaas, P. J. J., der Dulk-Ras, H. & Schilperoort, R. A. The Agrobacterium tumefaciens T-DNA gene 6b is an one gene. Plant Mol. Biol. 11, 791-794 (1988).
4. Spanier, K., Schell, J. & Schreier, P. H. A functional analysis of T-DNA gene 6b: the fine tuning of cytokinin effects on shoot development. Mol. Gen. Genet. 219, 209-216 (1989).
5. Tinland, B., Fournier, P., Heckel, T. & Otten, L. Expression of a chimeric heat shock-inducible Agrobacterium 6b oncogene in Nicotiana rustica. Plant Mol. Biol. 18, 921-930 (1992).
6. Wabiko, H. & Minemura, M. Exogenous phytohormone-independent growth and regeneration of tobacco plants transgenic for the 6b gene of Agrobacterium tumefaciens AKE10. Plant Physiol. 112, 939-951 (1996).
7. Gremillon, L., Helfer, A., Clement, B. & Otten, L. New plant growth-modifying properties of the Agrobacterium T-6b oncogene revealed by the use of a dexamethasone-inducible promoter. Plant J. 37, 218-228 (2004).
8. Kitakura, S. et al., The protein encoded by oncogene 6b from Agrobacterium tumefaciens interacts with a nuclear protein of tobacco. Plant Cell 14, 451-463 (2002).
9. Terakura S. et al. Oncogene 6b from Agrobacterium tumefaciens induces abaxial cell division at late stages of leaf development and modifies vascular development in petiole. Plant Cell Physiol. March 17 (2006).
10: Morris, R. O. Genes specifying auxin and cytokinin biosynthesis in phytopathogens. Annu. Rev. Plant Physiol. 37, 509-538 (1986)
11. Otten, L. & De Ruffray, P. Agrobacterium vitis nopaline Ti plasmid pTiAB4: relationship to other Ti plasmids and T-DNA structure. Mol. Gen. Genet. 245, 493-505 (1994).
12. Levesque, H., Delepelaire, P., Rouze, P., Slightom, J. & Tepfer, D. Common evolutionary origin of the central portions of the R1 TL-DNA of Agrobacterium rhizogenes and the Ti-DNAs of Agrobacterium tumefaciens. Plant Mol. Biol. 11, 731-744 (1988).
13. Spena, K., Schmuling, T., Konz, C. & Schell, J. S. Independent and synergistic activity of rol A, B, and C loci in stimulating abnormal growth in plants. EMBO J. 6, 3891-3899 (1987).
14. Leemans, J. et al. Broad-host-range cloning vectors derived from the W-plasmid Sa. Gene 19, 361-364 (1982).
15. Ream, L. W., Gordon, M. P. & Nester, E. W. Multiple mutations in the T region of the Agrobacterium tumefaciens tumor-inducing plasmid. Proc. Natl. Acad. Sci. USA. 80, 1660-1664 (1983).
16. Waterborg, J. H. & Robertson, A. J. Common features of analogous replacement histone H3 genes in animals and plants. J. Mol. Evol. 43, 194-206 (1996).
17. Helfer, A., Clement, B., Michler, P. & Otten, L. The Agrobacterium oncogene AB-6b causes a graft-transmissible enation syndrome in tobacco. Plant Mol. Biol. 52, 483-493 (2003).
18. Ray-Gallet, D. et al. HIRA is critical for a nucleosome-assembly pathway independent of DNA synthesis. Mol. Cell. 9, 1091-1100 (2002).
19. Munakata, T., Adachi, N., Yokoyama, N., Kuzuhara, T. & Horikoshi, M. A human homologue of yeast anti-silencing factor has histone-chaperone activity. Genes Cells 5, 221-223 (2000).
20. Ishimi, Y. & Kikuchi, A. Identification and molecular cloning of yeast homolog of nucleosome assembly protein which facilitates nucleosome assembly in vitro. J. Biol. Chem. 266, 7025-7029 (1991).
21. Okuwaki, M., Matsumoto, K., Tsujimoto, M. & Nagata, K. Function of nucleophosmin/B23, a nucleolar acidic protein, as a histone chaperone. FEBS Lett. 506, 272-276 (2001).
22. Kuzuhara, T. & Horikoshi, M. A nuclear FK506-binding protein is a histone chaperone regulating rDNA silencing. Nat. Struct. Mol. Biol. 11, 275-83 (2004).
23. Laskey, R. A., Honda, B. M., Mills, A. D. & Finch, J. T. nucleosomes are assembled by an acidic protein which binds histones and transfers them to DNA. Nature 275, 416-420 (1978).
24. Ishimi, Y., Yasuda, H., Hirosumi, J., Hanaoka, F. & Yamada, M. A protein which facilitates assembly of nucleosome-like structures in vitro in mammalian cells. J. Biochem. 94, 735-744 (1983)
25. Sealy, L., Burgess, R. R., Cotton, M. & Chalkley, R. Purification of Xenopus egg nucleoplasmin and its use in chromatin assembly in vitro. Methods Enzymol. 170, 612-630 (1989).
26. Loyola, A. & Almouzni, G. Histone chaperones, a supporting role in the limelight. Biochim. Biophys. Acta 1677, 3-11 (2004)
27. Zilberman, D. & Henikoff, S. Epigenetic inheritance in Arabidopsis: selective silence. Curr. Opin. Genet. Dev. 15, 557-62 (2005).
28. Ishikawa, M., Soyano, T., Nishihama, R. & Machida, Y. The NPK1 mitogenactivated protein kinase kinase kinase contains a functional nuclear localization signal at the binding site for the NACK1 kinesin-like protein. Plant J. 32, 789-798 (2002).
29. Masuda, K., Takahashi, S., Nomura, K. & Inoue, M. A simple procedure for the isolation of pure nuclei from carrot embryos in synchronized cultures. Plant Cell Reports 10, 329-333 (1991).
30. Simon, R. H. & Felsenfeld, G. A new procedure for purifying histone pairs H2A+H2B and H3+H4 from chromatin using hydroxylapatite. Nucleic Acids Res. 6, 689-696 (1979).

### INDUSTRIAL APPLICABILITY

This invention is useful for genetically modification of plant body and substance production by genetically modified plant body.

1. A modification method of modifying gene expression of a plant body, said modification method comprising at least one of the steps of:
(a) modifying the plant body to allow production of Agrobacterium 6b protein or a modified body thereof; and
(b) modifying the plant body to allow change in expression level of Agrobacterium 6b protein in an enhancing or repressing direction.

2. (canceled)

3. (canceled)

4. (canceled)

5. (canceled)

6. (canceled)

7. (canceled)

8. (canceled)

9. (canceled)

10. A modification method in accordance with claim 1, wherein the modification of Agrobacterium 6b protein includes modification of at least either of an amino acid sequence in an acidic amino acid region at a C-terminal and an amino acid sequence in an adjoining amino acid region adjacent to the acidic amino acid region at the C-terminal.

11. A modification method in accordance with claim 1, wherein the modification of Agrobacterium 6b protein includes modification of an amino acid sequence in an amino acid region other than the acidic amino acid region and the adjoining amino acid region at the C-terminal.

12. A modification method in accordance with claim 1, said method further comprising the steps of
(c) modifying the plant body to allow production of a histone protein or a modified body thereof which interacts with Agrobacterium 6b protein; and
(d) modifying the plant body to allow change in expression level of the histone protein in an enhancing or repressing direction.

13. A modification method in accordance with claim 12, wherein the histone protein is histone H3 protein.

14. A modification method in accordance with claim 13, wherein the modification of the histone protein includes modification of an amino acid sequence in at least either of a folding region and a non-folding region of histone H3 protein.

15. (canceled)

16. (canceled)

17. (canceled)

18. (canceled)

19. A plant body having modified gene expression, said plant body being modified to allow at least either of expression of Agrobacterium 6b protein or its modified body and change of an expression level of Agrobacterium 6b protein in an enhancing or repressing direction.

20. A plant body in accordance with claim 19, said plant body being further modified to allow at least either of expression of a histone protein or a modified body thereof, which interacts with Agrobacterium 6b protein, and change in expression level of the histone protein in an enhancing or repressing direction.

21. (canceled)

22. A gene expression method of expressing a gene of a plant body, said gene expression method comprising the steps of
(a) preparing a plant body modified to have a first modification and a second modification,
the first modification being attained either by enhancement or repression of an expression level of a selected internal gene or by introduction of an exogenous gene coding a selected protein,
the second modification being attained by at least either of expression of Agrobacterium 6b protein or its modified body and change of an expression level of Agrobacterium 6b protein in an enhancing or repressing direction; and
(b) proliferating, growing, or breeding the plant body.

23. (canceled)

24. (canceled)

25. (canceled)

26. A gene expression method in accordance with claim 22, wherein said step (a) introduces at least two different genes into to a host chromosome.

27. A gene expression method in accordance with claim 22, wherein the plant body expresses a modified body of histone H3 protein.

28. (canceled)

29. (canceled)

30. (canceled)

31. (canceled)

32. A plant chromatin structure having Agrobacterium 6b protein or a modified body thereof.

33. A plant chromatin structure in accordance with claim 32, said plant chromatin structure further having a histone protein or a modified body thereof, which interacts with Agrobacterium 6b protein.

34. (canceled)

35. A production method of producing a modified plant chromatin structure, said production method comprising the step of:
bringing Agrobacterium 6b protein or a modified body thereof in contact with a chromatin structure-producing material in a chromatin structure-producing environment.

36. A production method in accordance with claim 35, wherein said step bringing Agrobacterium 6b protein or the modified body thereof in contact with the chromatin structure-producing material in the presence of histone H3 protein in the chromatin

37. (canceled)


SEQUENCE LISTING (2 sequences)

SEQ ID NO: 1 - DNA, 627 bp, Agrobacterium tumefaciens
  CDS (1)..(627)
atgacggttcctacttggcaggttcgcgatcttagacgcatcctacgagttagtgagttgaggcaacacttgcgccaggctcgtaccgatttcagatccaccctgtcacaattcgtatactttaaccgctccgttgttaacccaaatgcgtacgatgacgagtatctcctttcagaccagaggctcacctatgtttacgtcgatgaagtaacagcgcaattgtgtggtctcaatcgcctattaccgtccaactctccggccttcgggacagtagctaccgcaatgcccccatggctattggatccacaagaaatgaacgcaatcttgcaacagagttgcggtcaaggaggcttcgttaattaccaccacggtccgagtacaaacagcttttttttggcgatcctgatgagccagttgttcatacggatcaggactgatgtaatccgcggtcaaggttatggttggtacgccaggctaggtaattatgttgaggaaggagaggataacgaaggtatcgaaaacgaggaagaggaagaggaagaggaagaggaaacgagagagttccaattatccgatctcattcattatcccattgtcgcattgggatcgtgccatctcacacgttag

SEQ ID NO: 2 - protein, 208 aa, Agrobacterium tumefaciens
MTVPTWQVRDLRRILRVSELRQHLRQARTDFRSTLSQFVYFNRSVVNPNAYDDEYLLSDQRLTYVYVDEVTAQLCGLNRLLPSNSPAFGTVATAMPPWLLDPQEMNAILQQSCGQGGFVNYHHGPSTNSFFLAILMSQLFIRIRTDVIRGQGYGWYARLGNYVEEGEDNEGIENEEEEEEEEEETREFQLSDLIHYPIVALGSCHLTR